(19) United States
(12) Patent Application Publication
(10) Pub. No.: US 2025/0090027 A1
(43) Pub. Date: Mar. 20, 2025

(54) MULTI-SENSOR DEVICES AND SYSTEMS

(71) Applicant: QUALCOMM Incorporated, San Diego, CA (US)

(72) Inventors: Hrishikesh Vijaykumar PANCHAWAGH, Cupertino, CA (US); Bernard HERRERA SOUKUP, Sunnyvale, CA (US); Emily Kathryn BROOKS, Amherst, NY (US); Nicholas BUCHAN, San Jose, CA (US); John Keith SCHNEIDER, Williamsville, NY (US); Kostadin Dimitrov DJORDJEV, Los Gatos, CA (US)

(21) Appl. No.: 18/468,616

(22) Filed: Sep. 15, 2023

(51) Int. Cl. A61B 5/00; A61B 5/02; A61B 5/021; G01P 15/09

(52) U.S. Cl. CPC A61B 5/0095; A61B 5/02007; A61B 5/02125; A61B 5/6801; A61B 5/7267; G01P 15/09; A61B 2562/0204; A61B 2562/0219

(57) ABSTRACT

Multi-sensor devices and systems are disclosed. A multi-sensor user device may include a first acoustic sensor and a second acoustic sensor disposed at a first distance from each other, the first and second acoustic sensors each configured to obtain respective first and second acoustic signals associated with a blood vessel of a user; a first motion sensor and a second motion sensor disposed at a second distance from each other, the first and second motion sensors each configured to obtain respective first and second motion signals associated with the blood vessel; a photoacoustic sensor configured to obtain a photoacoustic signal generated from light incident on the blood vessel; and a wearable structure securable to the user and comprising the first acoustic sensor, the second acoustic sensor, the first motion sensor, the second motion sensor, and the photoacoustic sensor.

### TECHNICAL FIELD

This disclosure relates generally to devices and systems using multiple types of sensors.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and/or ambulatory monitoring. Some such devices are, or include, photoacoustic sensors or optical sensors. Although some previously deployed devices can provide acceptable results, improved detection devices and systems would be desirable.

### SUMMARY

The systems, methods and devices of this disclosure each have several aspects, no single one of which is solely responsible for the desirable attributes disclosed herein.

In one aspect of the present disclosure, a multi-sensor user device is disclosed. In some embodiments, the multi-sensor user device may include: a first acoustic sensor and a second acoustic sensor disposed at a first distance from each other, the first and second acoustic sensors each configured to obtain respective first and second acoustic signals associated with a blood vessel of a user, the first distance corresponding to a first characteristic of the blood vessel; a first motion sensor and a second motion sensor disposed at a second distance from each other, the first and second motion sensors each configured to obtain respective first and second motion signals associated with the blood vessel, the second distance corresponding to a second characteristic of the blood vessel; a photoacoustic sensor configured to obtain a photoacoustic signal generated from light incident on the blood vessel, the photoacoustic signal corresponding to one or more physiological characteristics of the blood vessel, wherein a combination of the one or more physiological characteristics of the blood vessel, the first characteristic of the blood vessel, and the second characteristic of the blood vessel correlate to a physiological parameter of the user; and a wearable structure securable to the user and comprising the first acoustic sensor, the second acoustic sensor, the first motion sensor, the second motion sensor, and the photoacoustic sensor.

In another aspect of the present disclosure, a method of determining a physiological parameter of a user is disclosed. In some embodiments, the method may include: obtaining a first acoustic signal associated with a blood vessel of the user measured by a first acoustic sensor of a wearable device, and a second acoustic signal associated with the blood vessel measured by a second acoustic sensor of the wearable device which is disposed at a first distance from the first acoustic sensor; obtaining a first motion signal associated with the blood vessel measured by a first motion sensor of the wearable device, and a second motion signal associated with the blood vessel measured by a second motion sensor of the wearable device which is disposed at a second distance from the first motion sensor; obtaining a photoacoustic signal generated from light incident on the blood vessel measured by a photoacoustic sensor; and determining the physiological parameter of the user based on the first acoustic signal, the second acoustic signal, the first distance, the first motion signal, the second motion signal, the second distance, and the photoacoustic signal.

In another aspect of the present disclosure, an apparatus is disclosed. In some embodiments, the apparatus may include: first acoustic sensing means and second acoustic sensing means disposed at a first distance from each other, the first and second acoustic sensing means each being for obtaining respective first and second acoustic signals associated with a blood vessel of a user, the first distance corresponding to a first characteristic of the blood vessel; first motion sensing means and second motion sensing means disposed at a second distance from each other, the first and second motion sensing means each being for obtaining respective first and second motion signals associated with the blood vessel, the second distance corresponding to a second characteristic of the blood vessel; photoacoustic sensing means for obtaining a photoacoustic signal generated from light incident on the blood vessel, the photoacoustic signal corresponding to one or more physiological characteristics of the blood vessel, wherein a combination of the one or more physiological characteristics of the blood vessel, the first characteristic of the blood vessel, and the second characteristic of the blood vessel correlate to a physiological parameter of the user; and wearable means for securing the apparatus to the user and comprising the first acoustic sensing means, the second acoustic sensing means, the first motion sensing means, the second motion sensing means, and the photoacoustic sensing means.

In another aspect of the present disclosure, a non-transitory computer-readable apparatus is disclosed. In some embodiments, the non-transitory computer-readable apparatus may include a storage medium, the storage medium comprising a plurality of instructions configured to, when executed by one or more processors, cause an apparatus to: obtain a first acoustic signal associated with a blood vessel of a user measured by a first acoustic sensor of a wearable device, and a second acoustic signal associated with the blood vessel measured by a second acoustic sensor of the wearable device which is disposed at a first distance from the first acoustic sensor; obtain a first motion signal associated with the blood vessel measured by a first motion sensor of the wearable device, and a second motion signal associated with the blood vessel measured by a second motion sensor of the wearable device which is disposed at a second distance from the first motion sensor; obtain a photoacoustic signal generated from light incident on the blood vessel measured by a photoacoustic sensor; and determine a physiological parameter of the user based on the first acoustic signal, the second acoustic signal, the first distance, the first motion signal, the second motion signal, the second distance, and the photoacoustic signal.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 shows an example of a blood pressure monitoring device based on photoacoustic plethysmography, which may be referred to herein as PAPG.

FIG. 2 shows an example of a blood pressure monitoring device based on photoplethysmography (PPG).

FIG. 3 shows examples of heart rate waveform (HRW) features that may be extracted according to some implementations.

FIG. 4 shows examples of devices that may be used in a system for estimating blood pressure based, at least in part, on pulse transit time (PTT).

FIG. 5 shows a cross-sectional side view of a diagrammatic representation of a portion of an artery through which a pulse is propagating.

FIG. 6A shows an example monitoring device designed to be worn around a wrist according to some implementations.

FIG. 6B shows an example monitoring device designed to be worn on a finger according to some implementations.

FIG. 6C shows an example monitoring device designed to reside on an earbud according to some implementations.

FIG. 7 is a block diagram that shows example components of a PAPG-based multi-sensor apparatus according to some disclosed implementations.

FIG. 8 is a block diagram showing an example photoacoustic sensor system according to some disclosed implementations.

FIG. 9 is a block diagram showing an example acoustic sensor system according to some disclosed implementations.

FIG. 10 is a block diagram showing an example motion sensor system according to some disclosed implementations.

FIGS. 11A and 11B illustrate views of an example configuration of a PAPG-based multi-sensor apparatus, according to some embodiments.

FIG. 11C shows an example waveform that represents measurements by a photoacoustic sensor system used with a PAPG-based multi-sensor apparatus.

FIG. 11D shows example waveforms that represent measurements by acoustic sensor systems or motion sensor systems used with a PAPG-based multi-sensor apparatus.

FIGS. 12A-12C show example layouts of sensors of a PAPG-based multi-sensor apparatus.

FIG. 13 is a block diagram that shows example components of a PPG-based multi-sensor apparatus according to some disclosed implementations.

FIGS. 14A and 14B illustrate views of an example configuration of a PPG-based multi-sensor apparatus, according to some embodiments.

FIG. 14C shows an example waveform that represents measurements by an optical sensor system used with a PPG-based multi-sensor apparatus.

FIG. 14D shows example waveforms that represent measurements by acoustic sensor systems or motion sensor systems used with a PPG-based multi-sensor apparatus.

FIGS. 15A and 15B illustrate views of another example configuration of a PPG-based multi-sensor apparatus, according to some embodiments.

FIG. 15C shows example waveforms that represent measurements by optical sensor systems used with a PPG-based multi-sensor apparatus.

FIG. 15D shows example waveforms that represent measurements by acoustic sensor systems or motion sensor systems used with a PPG-based multi-sensor apparatus.

FIGS. 16A and 16B illustrate views of another example configuration of a PPG-based multi-sensor apparatus, according to some embodiments.

FIG. 16C shows example waveforms that represent measurements by optical sensor systems used with a PPG-based multi-sensor apparatus.

FIG. 16D shows example waveforms that represent measurements by acoustic sensor systems or motion sensor systems used with a PPG-based multi-sensor apparatus.

FIGS. 17A-17C show example layouts of sensors of a PPG-based multi-sensor apparatus.

FIG. 18 is a flow diagram of a method for determining a physiological parameter of a user, according to some disclosed implementations.

FIG. 19 is a flow diagram of another method for determining a physiological parameter of a user, according to some disclosed implementations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications or monitoring of other physiological parameters. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, chest bands, anklets, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

There is a strong need for accurate, non-invasive, continuous monitoring wearable devices for both clinical and consumer applications, e.g., for measuring physiological parameters such as blood pressure of a user. Measurement of arterial signals and heart rate waveforms from arteries is key to determining and predicting arterial (e.g., blood pressure) measurements. Non-invasive health monitoring devices, such as photoacoustic plethysmography (PAPG)-based devices, have various potential advantages over more invasive health monitoring devices such as cuff-based or catheter-based blood pressure measurement devices. Some wearable PAPG-based devices may include a platen or an interface for transmitting both light and acoustic signals. The platen or interface should be optically transparent and should ideally have an acoustic impedance that closely matches that of human skin. As discussed in more detail elsewhere herein, PAPG can measure various depth-discriminated artery waveforms and arterial characteristics such as diameter and pulse wave velocity, which in turn can be used to estimate blood pressure. Photoplethysmography (PPG) can also be used to monitor health of a user in a non-invasive way by transmitting light and receiving reflected light from a target object.

While PAPG and PPG alone can be useful and advantageous for non-invasive monitoring as mentioned so far, challenges still exist in obtaining accurate measurements from the target. For instance, blood pressure detection based on a single modality such as PAPG, PPG, acoustics, or pressure may not have sufficient information to result in sufficient resolution or quality of measurements of interest, especially arterial compliance and distension information. Further, a sensing modality from a single point of measurement may be sensitive to motion or environmental factors.

Hence, various aspects provided in the present disclosure relate generally to an approach that uses different sensing modalities to detect physiological parameters (including, e.g., blood pressure).

Some aspects more specifically relate to a multi-sensor device configured to obtain acoustic signals from two or more acoustic sensor systems (e.g., microphones), motion signals from two or more motion sensor systems (e.g., accelerometers), and photoacoustic signals from a photoacoustic sensor system (e.g., PAPG device). Alternate aspects relate to a device configured to obtain acoustic signals from two or more acoustic sensor systems (e.g., microphones), motion signals from two or more motion sensor systems (e.g., accelerometers), and photoacoustic signals from one or more optical sensor systems (e.g., PPG device(s)).

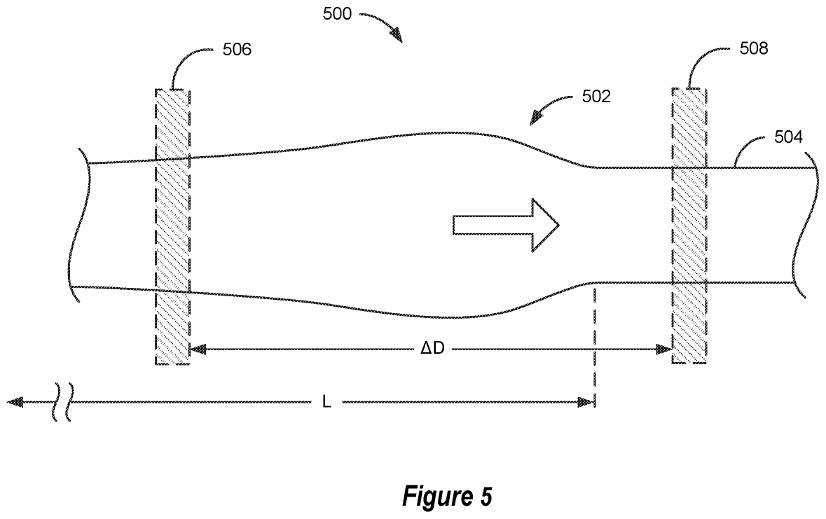

Such acoustic signals, motion signals, or photoacoustic (or optical) signals can be used to derive a physiological characteristic (e.g., pulse wave velocity, compliance, volume, size and deformation with time) of a target object. An example of the target object is a blood vessel of a user. The physiological characteristic can be used to derive a physiological parameter of the user (such as blood pressure). As an illustrative example, pulse wave velocity (PWV) can be determined based on the two acoustic sensors, the two motion sensors, or two optical sensors. As other examples, arterial diameter and/or distension can be determined based on the photoacoustic sensor. In turn, blood pressure can be estimated based on PWV, as discussed with respect to FIG. 5.

Further, in some implementations, machine learning can be used to train a machine learning model that can predict a physiological characteristic of a blood vessel (e.g., PTT, PWV) or parameter of a user (e.g., blood pressure), or examine such a physiological characteristic or parameter estimated using the multi-sensor device. Based on any discrepancies between the sensor-based estimation and the model-generated prediction, some or all of the sensor-based measurements can be kept or discarded.

Particular implementations of the subject matter described in this disclosure can be implemented to realize one or more of the following potential advantages. A combination of multiple sensing modalities can better determine and predict measurements that are more accurate than an individual modality alone (e.g., PAPG or PPG) while remaining non-invasive. Signals measured from multiple modalities within one multi-sensor device can corroborate or complement one another, as opposed to detecting acoustic signals by a PAPG sensor or optical signals by a PPG sensor. In some embodiments, (1) a photoacoustic sensor system (such as a PAPG sensor) or an optical sensor system (such as a PPG sensor), (2) an acoustic sensor system such as or including a microphone, and (3) a motion or inertial sensor system such as or including an accelerometer (e.g., voice accelerometer, contact microphone) can be deployed in the same device. These sensors are low power, low cost, and very compact, and possess high sensitivity and an ability to be mass produced to be integrated in various consumer devices, including wearable devices. These sensors can be configured to detect various characteristics, including, for example, artery diameter and distension, pressure, acoustic waves, volumetric blood flow, motion of blood vessel or tissue, and/or pulse wave velocity, while rejecting external disturbances and movement. In some embodiments, the multi-sensor device is implemented as a wearable device, enabling the foregoing advantages in a user-friendly fashion.

Additional details will follow after an initial description of relevant systems and technologies.

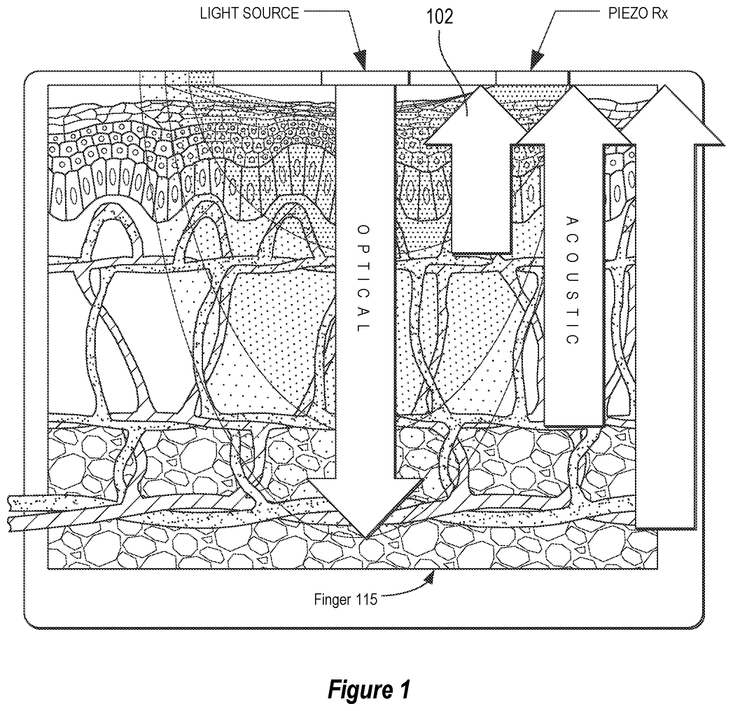

FIG. 1 shows an example of a blood pressure monitoring device based on photoacoustic plethysmography, which is referred to herein as PAPG. FIG. 1 shows the same examples of arteries, veins, arterioles, venules and capillaries inside a body part, which is a finger 115 in this example. In some examples, the light source shown in FIG. 1 may be coupled to a light source system (not shown) that is disposed remotely from the body part (e.g., finger 115). In some implementations, the light source may be an opening of an optical fiber or other waveguide. Such an opening may also be connected to an opening of an interface that is contactable with the body part. In some embodiments, the light source system may include one or more LEDs, one or more laser diodes, etc. In this example, the light source has transmitted light (in some examples, green, red, infrared, and/or near-infrared (NIR) light) that has penetrated the tissues of the finger 115 in an illuminated zone.

In the example shown in FIG. 1, blood vessels (and components of the blood itself) are heated by the incident light from the light source and are emitting acoustic waves 102. In this example, the emitted acoustic waves 102 include ultrasonic waves. According to this implementation, the acoustic wave emissions 102 are being detected by an ultrasonic receiver, which is a piezoelectric receiver in this example. Photoacoustic emissions 102 from the illuminated tissues, detected by the piezoelectric receiver, may be used to detect volumetric changes in the blood of the illuminated zone of the finger 115 that correspond to physiological data within the illuminated tissues of finger 115, such as heart rate waveforms. Although some of the tissue areas shown to be illuminated are offset from those shown to be producing photoacoustic emissions 102, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination.

One important difference between an optical technique such as a photoplethysmography (PPG)-based system the PAPG-based method of FIG. 1 is that the acoustic waves shown in FIG. 1 travel much more slowly than the reflected light waves involved in PPG. Accordingly, depth discrimination based on the arrival times of the acoustic waves shown in FIG. 1 is possible, whereas depth discrimination based on the arrival times of the light waves in PPG may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

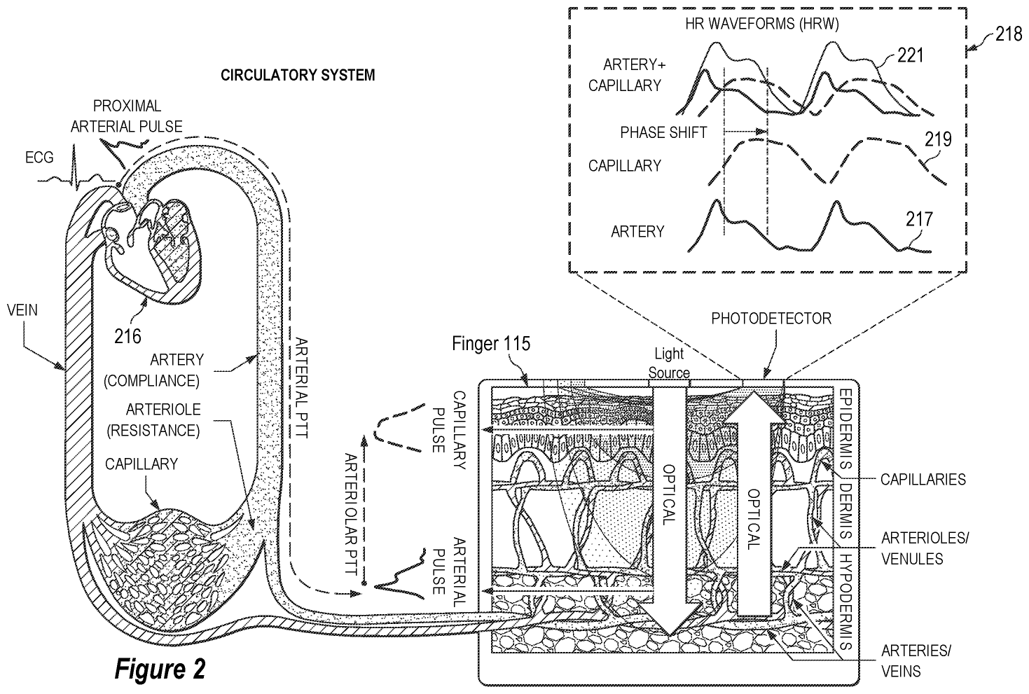

FIG. 2 shows an example of a blood pressure monitoring device based on photoplethysmography (PPG). FIG. 2 shows examples of arteries, veins, arterioles, venules and capillaries of a circulatory system, including those inside a finger 115. In the example shown in FIG. 2, an electrocardiogram (ECG) sensor has detected a proximal arterial pulse near the heart 216. Some examples are described below of measurement of the arterial pulse transit time (PTT) according to arterial pulses measured by two sensors, one of which may be an electrocardiogram sensor in some implementations.

According to the example shown in FIG. 2, a light source that includes one or more lasers or light-emitting diodes (LEDs) has transmitted light (in some examples, green, red, infrared, and/or near-infrared (NIR) light) that has penetrated the tissues of the finger 115 in an illuminated zone. Reflections from these tissues, detected by a photodetector, may be used to detect volumetric changes in the blood of the illuminated zone of the finger 115 that correspond to heart rate waveforms.

As shown in the heart rate waveform graphs 218 of FIG. 2, the capillary heart rate waveform 219 is differently-shaped and phase-shifted relative to the artery heart rate waveform 217. In this simple example, the detected heart rate waveform 221 is a combination of the capillary heart rate waveform 219 and the artery heart rate waveform 217. In some instances, the responses of one or more other blood vessels may also be part of the heart rate waveform 221 detected by a PPG-based blood pressure monitoring device.

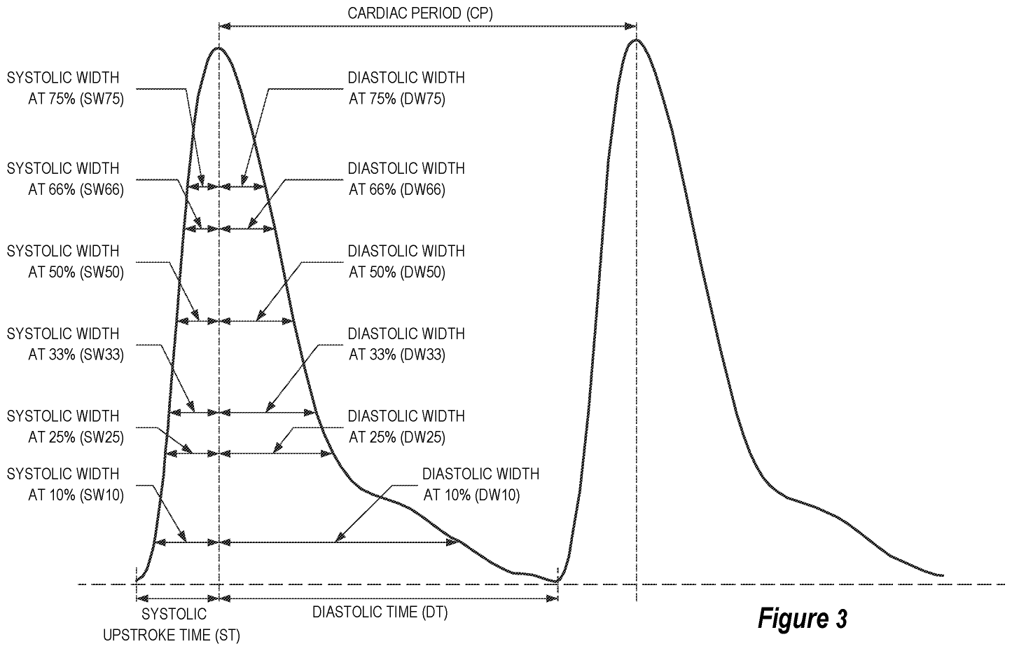

FIG. 3 shows examples of heart rate waveform (HRW) features that may be extracted according to some implementations. The horizontal axis of FIG. 3 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 3 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

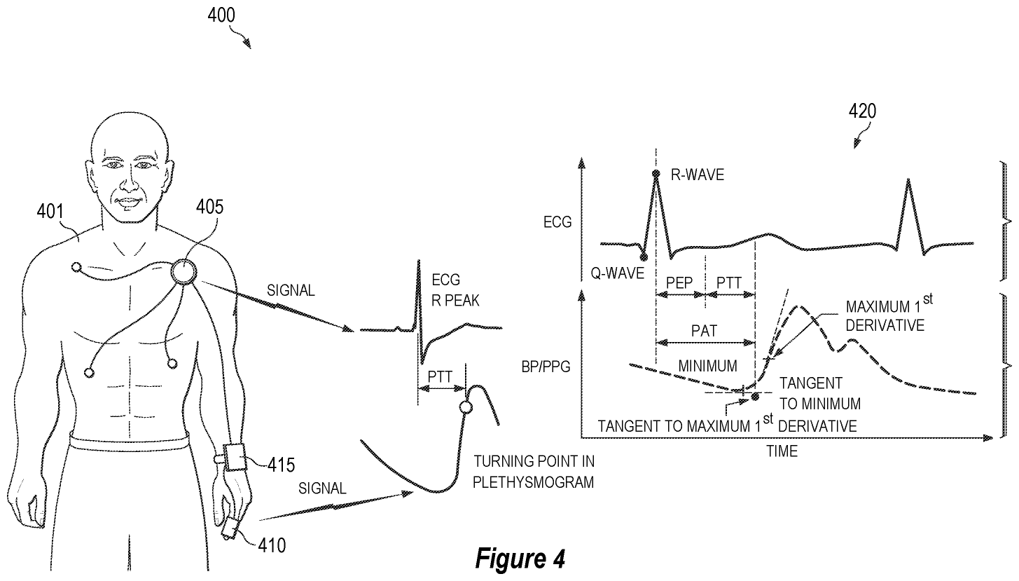

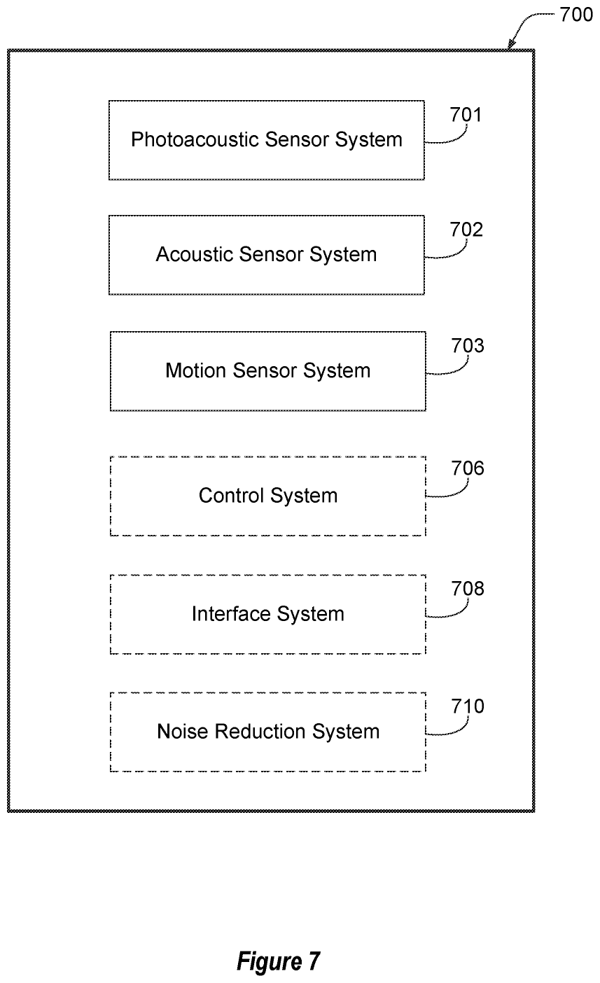

FIG. 4 shows examples of devices that may be used in a system for estimating blood pressure based, at least in part, on pulse transit time (PTT). As with other figures provided herein, the numbers, types and arrangements of elements are merely presented by way of example. According to this example, the system 400 includes at least two sensors. In this example, the system 400 includes at least an electrocardiogram sensor 405 and a device 410 that is configured to be mounted on a finger of the person 401. In this example, the device 410 is, or includes, an apparatus configured to perform at least some PAPG methods disclosed herein. For example, the device 410 may be, or may include, the apparatus 700 of FIG. 7 or a similar apparatus.

As noted in the graph 420, the PAT includes two components, the pre-ejection period (PEP), the time needed to convert the electrical signal into a mechanical pumping force and isovolumetric contraction to open the aortic valves) and the PTT. The starting time for the PAT can be estimated based on the QRS complex—an electrical signal characteristic of the electrical stimulation of the heart ventricles. As shown by the graph 420, in this example the beginning of a pulse arrival time (PAT) may be calculated according to an R-Wave peak measured by the electrocardiogram sensor 405 and the end of the PAT may be detected via analysis of signals provided by the device 410. In this example, the end of the PAT is assumed to correspond with an intersection between a tangent to a local minimum value detected by the device 410 and a tangent to a maximum slope/first derivative of the sensor signals after the time of the minimum value.

There are many known algorithms for blood pressure estimation based on the PTT and/or the PAT, some of which are summarized in Table 1 and described in the corresponding text on pages 5-10 of Sharma, M. et al., Cuff-Less and Continuous Blood Pressure Monitoring: a Methodological Review (“Sharma”), in Multidisciplinary Digital Publishing Institute (MDPI) Technologies 2017, 5, 21, both of which are hereby incorporated by reference.

Some previously-disclosed methods have involved calculating blood pressure according to one or more of the equations shown in Table 1 of Sharma, or other known equations, based on a PTT and/or PAT measured by a sensor system that includes a PPG sensor. As noted above, some disclosed PAPG-based implementations are configured to distinguish artery HRWs from other HRWs. Such implementations may provide more accurate measurements of the PTT and/or PAT, relative to those measured by a PPG sensor. Therefore, disclosed PAPG-based implementations may provide more accurate blood pressure estimations, even when the blood pressure estimations are based on previously-known formulae.

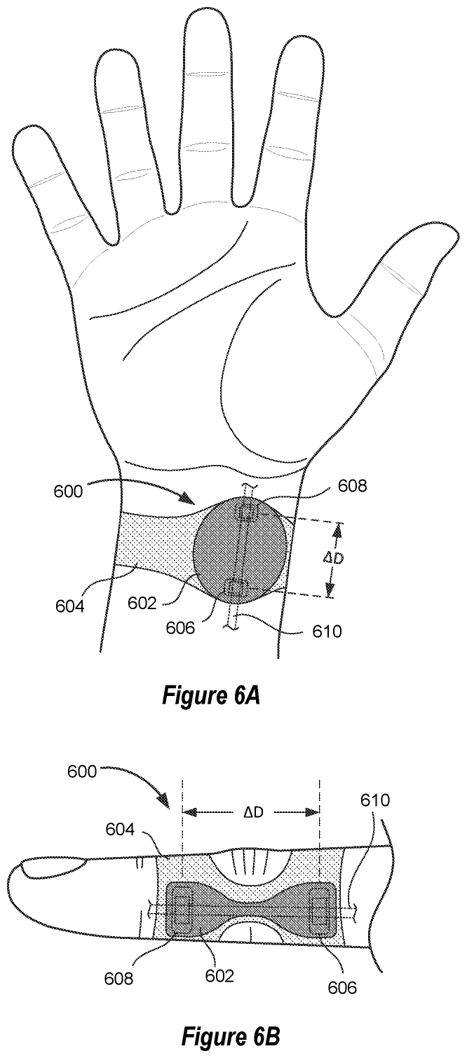

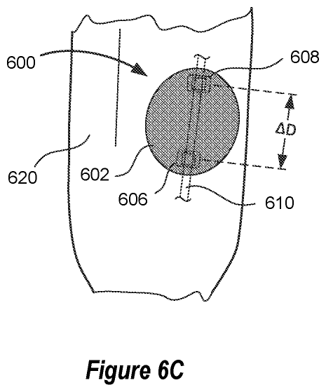

Other implementations of the system 400 may not include the electrocardiogram sensor 405. In some such implementations, the device 415, which is configured to be mounted on a wrist of the person 401, may be, or may include, an apparatus configured to perform at least some PAPG methods disclosed herein. For example, the device 415 may be, or may include, the apparatus 700 of FIG. 7 or a similar apparatus. According to some such examples, the device 415 may include a light source system and two or more ultrasonic receivers. One example is described below with reference to FIG. 6A. In some examples, the device 415 may include an array of ultrasonic receivers.

In some implementations of the system 400 that do not include the electrocardiogram sensor 405, the device 410 may include a light source system and two or more ultrasonic receivers. One example is described below with reference to FIG. 6B.

FIG. 5 shows a cross-sectional side view of a diagrammatic representation of a portion of an artery 500 through which a pulse 502 is propagating. The block arrow in FIG. 5 shows the direction of blood flow and pulse propagation. As diagrammatically shown, the propagating pulse 502 causes strain in the arterial walls 504, which is manifested in the form of an enlargement in the diameter (and consequently the cross-sectional area) of the arterial walls—referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length L of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. However, either version of the PTT may be used for the purpose of blood pressure estimation. Assuming that the physical distance ΔD between the first and the second physical locations is ascertainable, the PWV can be estimated as the quotient of the physical spatial distance ΔD traveled by the pulse divided by the time (PTT) the pulse takes in traversing the physical spatial distance ΔD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance ΔD through which the pulse propagates between the first physical location and the second physical location. More specifically, the PWV generally depends on a number of factors including the density of the blood ρ, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length ΔD between the two locations where the measurements are performed).

In traditional methods for obtaining PWV, the starting time of the pulse has been obtained at the heart using an electrocardiogram (ECG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 506 and a second arterial distension sensor 508 proximate first and second physical locations, respectively, along an artery of interest. In some particular implementations, the first arterial distension sensor 506 and the second arterial distension sensor 508 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance ΔD of separation between the first arterial distension sensor 506 and the second arterial distension sensor 508 (and consequently the distance between the first and the second locations along the artery) can be in the range of about 1 centimeter (cm) to tens of centimeters-long enough to distinguish the arrival of the pulse at the first physical location from the arrival of the pulse at the second physical location, but close enough to provide sufficient assurance of arterial consistency. In some specific implementations, the distance ΔD between the first and the second arterial distension sensors 506 and 508 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance ΔD between the first and the second arterial distension sensors 506 and 508 can be less than or equal to 1 cm, for example, about 0.1 cm, about 0.25 cm, about 0.5 cm or about 0.75 cm. By way of reference, a typical PWV can be about 15 meters per second (m/s). Using a monitoring device in which the first and the second arterial distension sensors 506 and 508 are separated by a distance of about 5 cm, and assuming a PWV of about 15 m/s implies a PTT of approximately 3.3 milliseconds (ms).

The value of the magnitude of the distance ΔD between the first and the second arterial distension sensors 506 and 508, respectively, can be preprogrammed into a memory within a monitoring device that incorporates the sensors (for example, such as a memory of, or a memory configured for communication with a control system such as 706 that is described below with reference to FIG. 7). As will be appreciated by a person of ordinary skill in the art, the spatial length L of a pulse can be greater than the distance ΔD from the first arterial distension sensor 506 to the second arterial distension sensor 508 in such implementations. As such, although the diagrammatic pulse 502 shown in FIG. 5 is shown as having a spatial length L comparable to the distance between the first arterial distension sensor 506 and the second arterial distension sensor 508, in actuality each pulse can typically have a spatial length L that is greater and even much greater than (for example, about an order of magnitude or more than) the distance ΔD between the first and the second arterial distension sensors 506 and 508.

In some implementations of the monitoring devices disclosed herein, both the first arterial distension sensor 506 and the second arterial distension sensor 508 are sensors of the same sensor type. In some such implementations, the first arterial distension sensor 506 and the second arterial distension sensor 508 are identical sensors. In such implementations, each of the first arterial distension sensor 506 and the second arterial distension sensor 508 utilizes the same sensor technology with the same sensitivity to the arterial distension signal caused by the propagating pulses, and has the same time delays and sampling characteristics. In some implementations, each of the first arterial distension sensor 506 and the second arterial distension sensor 508 is configured for photoacoustic plethysmography (PAPG) sensing, e.g., as disclosed elsewhere herein. Some such implementations include a light source system and two or more ultrasonic receivers. In some implementations, each of the first arterial distension sensor 506 and the second arterial distension sensor 508 is configured for ultrasound sensing via the transmission of ultrasonic signals and the receipt of corresponding reflections. In some alternative implementations, each of the first arterial distension sensor 506 and the second arterial distension sensor 508 may be configured for impedance plethysmography (IPG) sensing, also referred to in biomedical contexts as bioimpedance sensing. In various implementations, whatever types of sensors are utilized, each of the first and the second arterial distension sensors 506 and 508 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude- and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension.

In some implementations, regardless of the type of sensors utilized for the first arterial distension sensor 506 and the second arterial distension sensor 508, both the first arterial distension sensor 506 and the second arterial distension sensor 508 can be arranged, assembled or otherwise included within a single housing of a single monitoring device. As described above, the housing and other components of the monitoring device can be configured such that when the monitoring device is affixed or otherwise physically coupled to a subject, both the first arterial distension sensor 506 and the second arterial distension sensor 508 are in contact with or in close proximity to the skin of the user at first and second locations, respectively, separated by a distance ΔD, and in some implementations, along a stretch of the artery between which various arterial properties can be assumed to be relatively constant. In various implementations, the housing of the monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the monitoring device facilitates and enables long-term wearing and monitoring (for example, over days, weeks or a month or more without interruption) of one or more biological characteristics of interest to obtain a better picture of such characteristics over extended durations of time, and generally, a better picture of the user's health.

In some implementations, the monitoring device can be positioned around a wrist of a user with a strap or band, similar to a watch or fitness/activity tracker. FIG. 6A shows an example monitoring device 600 designed to be worn around a wrist according to some implementations. In the illustrated example, the monitoring device 600 includes a housing 602 integrally formed with, coupled with or otherwise integrated with a wristband 604. The first and the second arterial distension sensors 606 and 608 may, in some instances, each include an instance of the ultrasonic receiver system and a portion of the light source system that are described above. In this example, the monitoring device 600 is coupled around the wrist such that the first and the second arterial distension sensors 606 and 608 within the housing 602 are each positioned along a segment of the radial artery 610 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 606 and 608 are separated by a fixed distance ΔD. In some other implementations, the monitoring device 600 can similarly be designed or adapted for positioning around a forearm, an upper arm, an ankle, a lower leg, an upper leg, or a finger (all of which are hereinafter referred to as “limbs”) using a strap or band.

FIG. 6B shows an example monitoring device 600 designed to be worn on a finger according to some implementations. The first and the second arterial distension sensors 606 and 608 may, in some instances, each include an instance of the ultrasonic receiver and a portion of the light source system that are described above.

In some other implementations, the monitoring devices disclosed herein can be positioned on a region of interest of the user without the use of a strap or band. For example, the first and the second arterial distension sensors 606 and 608 and other components of the monitoring device can be enclosed in a housing that is secured to the skin of a region of interest of the user using an adhesive or other suitable attachment mechanism (an example of a “patch” monitoring device).

FIG. 6C shows an example monitoring device 600 designed to reside on an earbud according to some implementations. According to this example, the monitoring device 600 is coupled to the housing of an earbud 620. The first and second arterial distension sensors 606 and 608 may, in some instances, each include an instance of the ultrasonic receiver and a portion of the light source system that are described above.

### Example Multi-Sensor PAPG Apparatus

FIG. 7 is a block diagram that shows example components of a PAPG-based multi-sensor apparatus according to some disclosed implementations. In this example, the apparatus 700 may include a photoacoustic sensor system 701, an acoustic sensor system 702, and a motion sensor system 703. Some implementations of the apparatus 700 may include a control system 706, an interface system 708, a noise reduction system 710, or combinations thereof. In specific implementations, the interface system 708 (including, e.g., a contact surface) may be included so as to allow contact with skin to maximize the sensitivity of the acoustic sensor system 702 and/or the motion sensor system 703.

Various configurations of the photoacoustic sensor system 701, acoustic sensor system 702, and motion sensor system 703 are disclosed herein. Specific examples and implementations are described in more detail below.

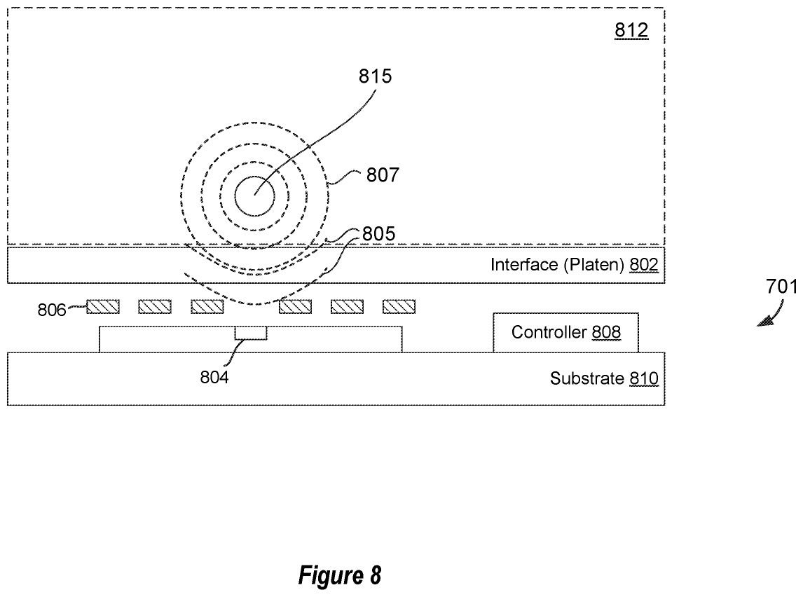

In some embodiments, the photoacoustic sensor system 701 may include an interface, a light source system, a receiver system, and may be an example of the blood pressure monitoring device based on PAPG as shown in FIG. 1. In some implementations, the photoacoustic sensor system 701 may also include a controller system or a controller. An example configuration of these components of the photoacoustic sensor system 701 is shown in FIG. 8.

Some disclosed PAPG sensors described herein (such as photoacoustic sensor system 701) may include a platen, a light source system, and an ultrasonic receiver system. According to some implementations, the light source system may include a light source configured to produce and direct light. In some implementations, the platen may include an anti-reflective layer, a mirror layer, or combinations thereof. According to some implementations, the platen may have an outer surface, or a layer on the outer surface, with an acoustic impedance that is configured to approximate the acoustic impedance of human skin. In some implementations, the platen may have a surface proximate the ultrasonic receiver system, or a layer on the surface proximate the ultrasonic receiver system, with an acoustic impedance that is configured to approximate the acoustic impedance of the ultrasonic receiver system.

Some disclosed PAPG sensors described herein (such as photoacoustic sensor system 701) may include an interface, a light source system and an ultrasonic receiver system. Some such devices may not include a rigid platen. According to some implementations, the interface may be a physical, flexible interface constructed of one or more of suitable materials having a desired property or properties (e.g., an acoustic property such as acoustic impedance, softness of the material). In some implementations, the interface may be a flexible interface that can contact a target object that may be proximate to or contact the interface. There may be salient differences between such an interface and a platen. In some implementations, the light source system may be configured to direct light using one or more optical waveguides (e.g., optical fibers) configured to direct light toward a target object. According to some implementations, the interface may have an outer surface, or a layer on the outer surface, with an acoustic impedance that is configured to approximate the acoustic impedance of human skin. Such outer surface may have a contact portion that is contactable by a user or a body part of the user (e.g., finger, wrist). In some examples, the optical waveguide(s) may be embedded in one or more acoustic matching layers that are configured to bring the light transmitted by the optical waveguide(s) very close to tissue. The outer surface and/or other parts of the interface may be compliant, pliable, flexible, or otherwise at least partially conforming to the shape and contours of the body part of the user. In some implementations, the interface may have a surface proximate the ultrasonic receiver system, or a layer on the surface proximate the ultrasonic receiver system, with an acoustic impedance that is configured to approximate the acoustic impedance of the ultrasonic receiver system.

FIG. 8 depicts an interface that is a platen 802 that allows transmission of optical signals (e.g., light) and acoustic signals. The platen 802 may be fixed relative to other components including a support structure such as a housing (not shown) and/or a substrate 810. The platen 802 may be an interface having a contact portion configured to make contact with a body part 812 of a user (such as the finger 115 shown in FIG. 1). The platen 802 may be contactable with tissue such as skin of a user such that there is good acoustic coupling for transmission of acoustic signals. The material of the platen 802 as well as other characteristics (e.g., thickness) may be selected to optimize acoustic transmission efficiency or signal quality (e.g., signal-to-noise ratio (SNR)). According to some examples, the platen 802 (or another portion of the apparatus 200) may include one or more anti-reflective layers. In some implementations, a coating or at least one additional layer may be disposed on the platen 802 to minimize noise. In some examples, one or more anti-reflective layers may reside on portions of an outer surface of the platen 802. Relatedly, in some implementations, the apparatus 700 may include a noise reduction system 710, as will be discussed below.

In some examples, at least a portion of the outer surface of the platen 802 may have an acoustic impedance that is configured to approximate an acoustic impedance of human skin. A typical range of acoustic impedances for human skin is 1.53-1.680 MRayls. Hence, in some examples, at least an outer surface of the platen 802 may have an acoustic impedance that is in the range of 1.4-1.8 MRayls, or in the range of 1.5-1.7 MRayls.

In some embodiments, such as that shown in FIG. 8, the light source system may include one or more light sources (e.g., light source 804) that may be disposed below the platen 802 and one or more receiver elements 806 of a receiver system. In some implementations, some or all of the one or more light sources may be disposed more remotely from the platen 802 and/or the one or more receiver elements 806. For example, a light source may be in a portion of a wearable device that is opposite to the platen 802 and the one or more receiver elements 806, and an optical waveguide (e.g., optical fiber) may be coupled between the remote light source and the platen 802. In such cases, light or other optical signals may travel via the optical waveguide toward a target object (e.g., artery 815) that is proximate to the platen 802.

In some embodiments, the light source system may, include one or more light-emitting diodes. In some implementations, the light source system may include one or more laser diodes. According to some implementations, the light source system may include one or more vertical-cavity surface-emitting lasers (VCSELs). In some implementations, the light source system may include one or more edge-emitting lasers. In some implementations, the light source system may include one or more neodymium-doped yttrium aluminum garnet (Nd:YAG) lasers.

Hence, the light source 804 may be, for example, a laser diode, a light-emitting diode (LED), or an array of either or both. The light source 804 may be configured to generate and emit optical signals 805. The light source system may, in some examples, be configured to transmit light in one or more wavelength ranges. In some examples, the light source system may be configured to transmit light in a wavelength range of 500 to 600 nanometers (nm). According to some examples, the light source system may be configured to transmit light in a wavelength range of 800 to 950 nm. According to some examples, the light source system may be configured to transmit light in infrared or near infrared (NIR) region of the electromagnetic spectrum (about 700 to 2500 nm). In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm, or about 700 to 900 nm, or about 600 to 1100 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist. In some implementations, the light source system or light source 804 may be configured to switch wavelengths to capture acoustic information from different depths, e.g., based on signal(s) from a controller 808 of the photoacoustic sensor system 701.

In some implementations, the light source system may be configured for emitting various wavelengths of light, which may be selectable to trigger acoustic wave emissions primarily from a particular type of material. That is, light sources may correspond to visible light, infrared light, or both. For example, because the hemoglobin in blood absorbs near-infrared light very strongly, in some implementations the light source system may be configured for emitting one or more wavelengths of light in the near-infrared range, in order to trigger acoustic wave emissions from hemoglobin. However, in some examples, the controller 808 and/or control system 706 may control the wavelength(s) of light emitted by the light source system to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying range gate delays (RGDs)) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system may be configured for emitting a plurality of light pulses at a pulse repetition frequency between about 1 MHz and about 100 MHz. Alternatively, or additionally, in some implementations the light source system may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

According to some examples, the light source system may also include one or more light-directing elements configured to direct light from the light source system towards the target object along the first axis. In some examples, the one or more light-directing elements may include at least one diffraction grating. Alternatively, or additionally, the one or more light-directing elements may include at least one lens.

In some example implementations, some or all of the one or more light sources may be disposed at or along an axis that is parallel to or angled relative to a central axis associated with the interface or platen 802. FIG. 8 shows optical signals 805 emitted toward the target object (e.g., artery 815), which may cause generation of ultrasonic waves 807 by the target object. These ultrasonic waves 807 may be detectable by one or more receiver elements 806.

In various configurations, the light source 804 may incorporate anti-reflection (AR) coating, a mirror, a light-blocking layer, a shield to minimize crosstalk, etc.

The light source system may include various types of drive circuitry, depending on the particular implementation. In some disclosed implementations, the light source system may include at least one multi-junction laser diode, which may produce less noise than single-junction laser diodes. In some examples, the light source system may include a drive circuit (also referred to herein as drive circuitry) configured to cause the light source system to emit pulses of light at pulse widths in a range from 3 nanoseconds to 1000 nanoseconds. According to some examples, the light source system may include a drive circuit configured to cause the light source system to emit pulses of light at pulse repetition frequencies in a range from 1 kilohertz to 100 kilohertz.

Various examples of a receiver system are disclosed herein, some of which may include ultrasonic receiver systems, optical receiver systems, or combinations thereof. In some implementations, the receiver system includes an ultrasonic receiver system having the one or more receiver elements 806. In implementations that include an ultrasonic receiver system, the ultrasonic receiver and an ultrasonic transmitter may be combined in an ultrasonic transceiver. In some examples, the receiver system may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system may be, or may include, an ultrasonic receiver array. In some examples, the photoacoustic sensor system 701 may include one or more separate ultrasonic transmitter elements or one or more separate arrays of ultrasonic transmitter elements. In some examples, the ultrasonic transmitter(s) may include an ultrasonic plane-wave generator.

In some implementations, at least portions of the photoacoustic sensor system 701 (for example, the receiver system, the light source system, or both) may include one or more sound-absorbing layers, acoustic isolation material, light-absorbing material, light-reflecting material, or combinations thereof. In some examples, acoustic isolation material may reside between the light source system and at least a portion of the receiver system. In some examples, at least portions of the photoacoustic sensor system 701 (for example, the receiver system, the light source system, or both) may include one or more electromagnetically shielded transmission wires. In some such examples, the one or more electromagnetically shielded transmission wires may be configured to reduce electromagnetic interference from the light source system that is received by the receiver system.

The controller 808 and/or the control system 706 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The controller 808 and/or the control system 706 also may include (and/or be configured for communication with) one or more memory devices, such as one or more random access memory (RAM) devices, read-only memory (ROM) devices, etc. Accordingly, the photoacoustic sensor system 701 may have a memory system that includes one or more memory devices, though the memory system is not shown in FIG. 8. The controller 808 and/or the control system 706 may be configured for receiving and processing data from the receiver system, e.g., as described below. If the photoacoustic sensor system 701 includes an ultrasonic transmitter, the controller 808 and/or the control system 706 may be configured for controlling the ultrasonic transmitter. In some implementations, functionality of the controller 808 and/or the control system 706 may be partitioned between one or more controllers or processors, such as a dedicated sensor controller and an applications processor of a mobile device.

In some examples, the controller 808 and/or the control system 706 may be communicatively coupled to the light source system and configured to control the light source system (including light source 804) to emit light towards a target object on an outer surface of the interface (e.g., platen 802). In some such examples, the controller 808 and/or the control system 706 may be configured to receive signals from the ultrasonic receiver system (including one or more receiver elements 806) corresponding to the ultrasonic waves generated by the target object responsive to the light from the light source system. In some examples, the controller 808 and/or the control system 706 may be configured to identify one or more blood vessel signals, such as arterial signals or vein signals, from the ultrasonic receiver system. In some such examples, the one or more arterial signals or vein signals may be, or may include, one or more blood vessel wall signals corresponding to ultrasonic waves generated by one or more arterial walls or vein walls of the target object. In some such examples, the one or more arterial signals or vein signals may be, or may include, one or more arterial blood signals corresponding to ultrasonic waves generated by blood within an artery of the target object or one or more vein blood signals corresponding to ultrasonic waves generated by blood within a vein of the target object. In some examples, the controller 808 and/or the control system 706 may be configured to determine or estimate one or more physiological parameters or cardiac features based, at least in part, on one or more arterial signals, on one or more vein signals, or on combinations thereof. According to some examples, the cardiac features may be, or may include, blood pressure. In some embodiments, the controller 808 and/or the control system 706 may communicate data such as the physiological parameters or cardiac features with a control system 706 of the apparatus 700, and the control system 706 may use the data from the photoacoustic sensor system 701 along with data from other components of the apparatus 700, e.g., the acoustic sensor system 702 and the motion sensor system 703 to determine enhanced physiological parameters according to various embodiments described herein.

In further examples, the controller 808 and/or the control system 706 may be communicatively coupled to the receiver system. The receiver system may be configured to detect acoustic signals from the target object. The controller 808 and/or the control system 706 may be configured to select at least one of a plurality of receiver elements 806 of the receiver system. Such selected receiver element(s) 806 may correspond to the best signals from multiple receiver elements. In some embodiments, the selection of the at least one receiver element may be based on information regarding detected acoustic signals (e.g., arterial signals or vein signals) from the plurality of receivers. For example, signal quality or signal strength (based, e.g., on signal-to-noise ratio (SNR)) of some signals may be relatively higher than some others or above a prescribed threshold or percentile, which may indicate the best signals. In some implementations, the controller 808 and/or the control system 706 may also be configured to, based on the information regarding detected acoustic signals, determine or estimate at least one characteristic of the blood vessels such as pulse wave velocity (indicative of arterial stiffness), arterial dimensions, or both.

Some implementations of the apparatus 700 may include an interface system 708. In some examples, the interface system 708 may include a wireless interface system. In some implementations, the interface system 708 may include a user interface system, one or more network interfaces, one or more interfaces between the controller 808 and/or the control system 706 and a memory system and/or one or more interfaces between the controller 808 and/or the control system 706 and one or more external device interfaces (e.g., ports or applications processors), or combinations thereof. According to some examples in which the interface system 708 is present and includes a user interface system, the user interface system may include a microphone system, a loudspeaker system, a haptic feedback system, a voice command system, one or more displays, or combinations thereof. According to some examples, the interface system 708 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 708 may include a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some implementations, the force sensor system may be mechanically coupled to the platen 802. In some such examples, the force sensor system may be integrated into circuitry of the ultrasonic fingerprint sensor. In some examples, the interface system 708 may include an optical sensor system, one or more cameras, or a combination thereof.

According to some examples, the apparatus 700 may include a noise reduction system 710. For example, the noise reduction system 710 may include one or more mirrors that are configured to reflect light from the light source system away from the receiver system. In some implementations, the noise reduction system 710 may include one or more sound-absorbing layers, acoustic isolation material, light-absorbing material, light-reflecting material, or combinations thereof. In some examples, the noise reduction system 710 may include acoustic isolation material, which may reside between the light source system and at least a portion of the receiver system, on at least a portion of the receiver system, or combinations thereof. In some examples, the noise reduction system 710 may include one or more electromagnetically shielded transmission wires. In some such examples, the one or more electromagnetically shielded transmission wires may be configured to reduce electromagnetic interference from circuitry of the light source system, receiver system circuitry, or combinations thereof, that is received by the receiver system.

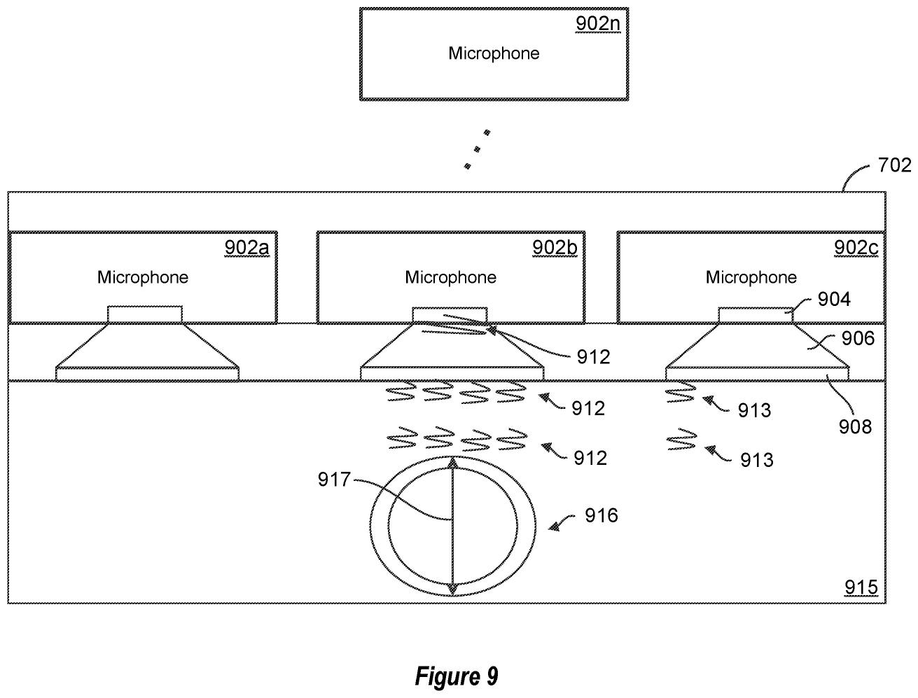

In some embodiments, the acoustic sensor system 702 may include one or more microphone elements. An example configuration of the acoustic sensor system 702 is shown in FIG. 9. As depicted in FIG. 9, embodiments of the acoustic sensor system 702 may include a plurality of microphones 902a-902c. Each microphone may be a MEMS microphone having an inlet port 904, a cavity 906, and a membrane or mesh 908 to facilitate detection and receipt of acoustic signals (e.g., sound waves 912) from the body part 915 of the user (such as the finger 115 shown in FIG. 1). Microphones may be coupled using a gasket creating cavities to amplify sound. The membrane or mesh 908 may be a thin layer that separates the cavity 906 and inlet port 904 from the skin, and protects the microphone components from dust, sweat, etc.

In an example scenario, a blood vessel 916 may generate acoustic signals. Pressure waves may be created by blood pressure within the blood vessel 916 and/or variations in blood pressure and associated movements (e.g., distension or contraction 917) of the blood vessel 916. The pressure waves can produce sound waves 912 in the tissue of the body part 915, which may be detected by the microphone via the membrane or mesh 908, the cavity 906, and then the inlet port 904.

Various configurations and designs of a microphone and its components (e.g., 904-908) may be envisioned by those having ordinary skill in the relevant arts. For instance, the shape of the cavity 906 may be narrower or wider depending on the configuration. Example dimensions of a microphone may be 3 mm by 3 mm in size. A microphone may provide digital or analog signals measuring sound pressure level (SPL) at the inlet port 904. Heart rate waveforms can have relatively low frequencies (e.g., under 20 Hz). To detect sound waves from the blood vessel 916, sensitivity of the microphone may be very high, e.g., able to detect low frequencies at 1-10 Hz. Since some microphones can detect dynamic signals, heart rate signals detected by microphones can also appear as “high pass” signals of true heart rate waveforms filtering very low frequencies.

Depending on the configuration, a microphone may be based on capacitive, piezoelectric, and/or other sensing modalities. Examples may include a piezoelectric MEMS microphone or a capacitive MEMS microphone.

As shown in FIG. 9, multiple microphones 902a-902c may be arranged in a substantially orthogonal direction as the blood vessel 916 which extends into and out of the illustration. An array of microphones can be used to achieve area diversity. This may enable maximal coverage to detect sound waves 912. For example, microphone 902c may pick up additional sound waves 913 from the blood vessel 916, while the majority of sound waves may be detected by microphone 902b. Sound waves 912, 913 may having sufficient quality or signal strength (e.g., meeting a threshold) may be used or transmitted (e.g., to the control system 706) while other signals are discarded.

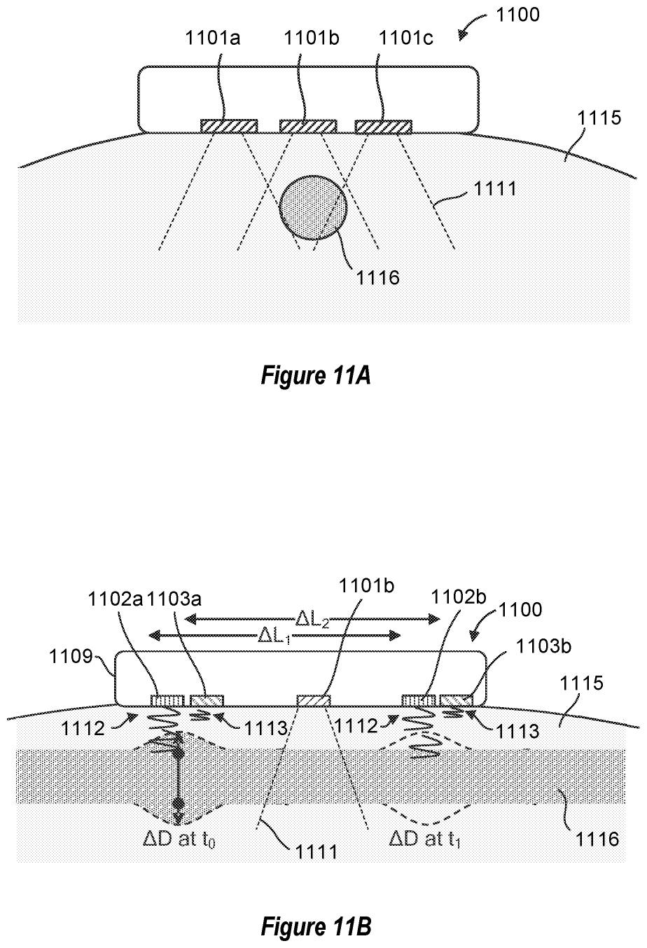

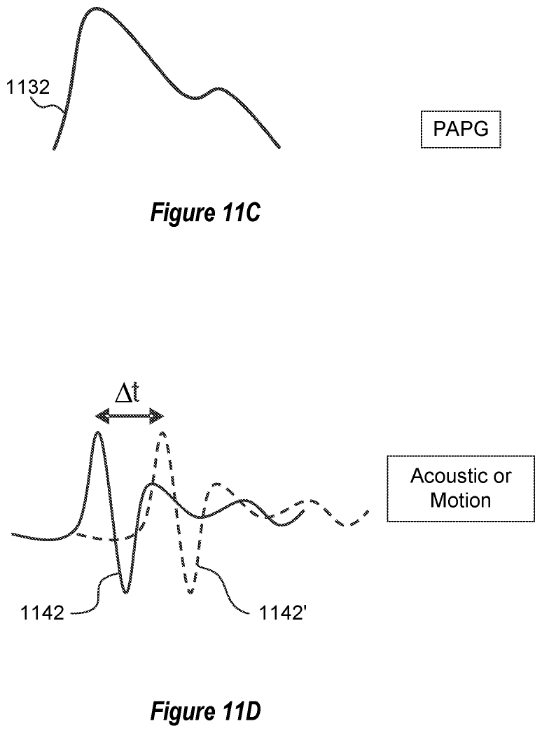

In some embodiments, at least one additional microphone 902n may be disposed along the blood vessel 916 at a distance from another microphone (e.g., 902b). This may allow measurements to be taken with a time delay, which is useful for estimating parameters such as pulse wave velocity, as will be described in further detail below with respect to FIGS. 11B and 11D, for example.

In certain embodiments, an acoustic sensor system may include filters such as a low-pass filter configured to remove noise sources in higher frequencies (e.g., over 20 Hz from noise or motion-related artifacts) and support low-frequency acoustic signals. In some cases, a high-pass filter may be used to filter out lower frequencies of acoustic signals instead (e.g., under 1 Hz from motion or other sources such as breathing). In addition, digital signal processing (e.g., in a built-in ASIC) may be used by an acoustic sensor system to improve the quality of acoustic signals.

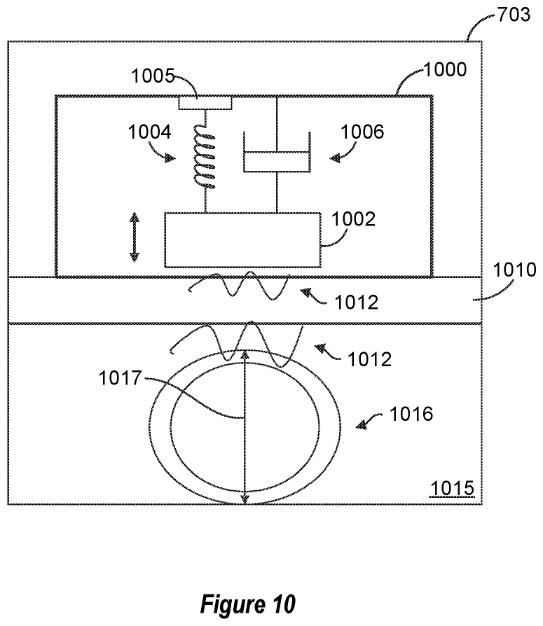

FIG. 10 is a block diagram showing an example motion sensor system 703 according to some disclosed implementations. In some embodiments, the motion sensor system 703 may include a spring-mass accelerometer 1000 comprising a spring 1004 having a known spring constant and a damper 1006 each coupled to the mass 1002. Movement of the mass 1002 may be restrained by the damper 1006. Some configurations may include multiple springs. Vibration or acceleration resulting from motion (e.g., distension or contraction 1017) of a blood vessel 1016 (and thereby tissue and skin) in a user's body part 1015 (such as the finger 115) may produce motion signals-waves 1012 of sound or motion of blood vessel 1016, skin, and/or body part 1015. These waves may push against the mass 1002 of the motion sensor system 703. Change in motion (acceleration) may cause the mass 1002 to exert force to a piezoelectric sensor 1005 that produces an electrical charge proportional to the force. Since the electrical charge is proportional to the force and the mass is constant, the charge is also proportional to the acceleration. A measurement of the compression of the spring 1004 (e.g., using a position sensor) may also be a measurement of acceleration.

In some example implementations, the motion sensor system 703 may be implemented as an accelerometer such as a MEMS voice accelerometer. Such an accelerometer may detect vibrations, motion, acceleration of the surface 1010 it is in contact with, e.g., skin of the user. In other embodiments, the motion sensor system 703 may include an inertial sensor (e.g., gyroscope). The motion sensor system 703 could also be used to obtain additional information about tissue motion, cancel environmental noise or motion, and improve signal quality and enhance physiological measurements relating to heart rate, blood pressure, etc.

Various configurations and designs of an accelerometer and its components (e.g., 1002-1006) may be envisioned by those having ordinary skill in the relevant arts. Depending on the configuration, accelerometers may be based on capacitive, piezoelectric, and/or other sensing modalities. Example dimensions of an accelerometer may be 3 mm by 3 mm in size, and may provide digital or analog signals measuring motion (e.g., motion of tissue) using a mass-spring-damper system as shown in FIG. 10. To detect sound waves from the blood vessel 1016, sensitivity of the accelerometer may be very high, e.g., able to detect low frequencies at 1-10 Hz. An array of accelerometer can be used to achieve area diversity. Multiple accelerometers may be positioned orthogonal to the direction of the blood vessel 1016 or along the blood vessel 1016 at a distance from another accelerometer. This may allow measurements to be taken with a time delay, which is useful for estimating parameters such as pulse wave velocity, as will be described in further detail below with respect to FIGS. 11B and 11D, for example.

In certain embodiments, acoustic sensor systems (e.g., microphones) may be configured to enable a wake-up functionality for the apparatus 700 or its components. For example, the control system 706 (and/or the controller 808 in the case of the photoacoustic sensor system) may be configured to wake up one or more motion sensor system and/or the photoacoustic sensor system from a low-power state responsive to one or more of the acoustic sensor systems detecting an acoustic signal having a signal quality above a threshold. When a microphone detects a good signal, it may be indicative of a steady environment without excessive background noise. Hence, in certain embodiments, one or more acoustic sensor systems may always be on or active, listening for signals, while other sensor systems (possibly including one or more of the acoustic sensor systems) are in a low-power or sleep state until woken up.

The apparatus 700 may be used in a variety of different contexts, many examples of which are disclosed herein. For example, in some implementations a mobile device may include the photoacoustic sensor system 701, the acoustic sensor system 702, and the motion sensor system 703. In some such examples, the mobile device may be a smart phone. In some implementations, a wearable device may include the photoacoustic sensor system 701, the acoustic sensor system 702, and the motion sensor system 703. The wearable device may, for example, be a bracelet, an armband, a wristband, a watch, a ring, a headband or a patch. In certain embodiments, the wearable device may be one or a pair of earbuds, a headset, a head rest mount, a headband, headphones, or another head-wearable or head-mounted device. An example monitoring device designed to reside on an earbud is shown in FIG. 6C.

### Example Configurations of PAPG Apparatus

FIG. 11A shows one view of an example configuration of a PAPG-based multi-sensor apparatus 1100, according to some embodiments. The PAPG-based multi-sensor apparatus 1100 may be an example of apparatus 700, and may have components including one or more photoacoustic sensor systems 1101a-1101c, a plurality of acoustic sensor systems 1102a, 1102b, and a plurality of motion sensor systems 1103a, 1103b. This view shows a cross-sectional view of a body part 1115 of a user (e.g., finger 115) where the photoacoustic sensor systems 1101a-1101c are located. In some implementations (as shown in FIG. 11B), the foregoing components may abut a surface of a housing structure 1109, where the surface is contactable with the body part 1115 of the user (e.g., via a contact surface of an interface system 708) to maximize sensitivity.

Each of photoacoustic sensor systems 1101a-1101c may be an example of photoacoustic sensor system 701, and hence may be configured to emit optical signals (e.g., light 1111) and detect acoustic signals that result from a target object or tissue of interest, such as blood vessel 1116 of the body part 1115 of the user, according to principles described with respect to FIG. 1. Each photoacoustic sensor system may be configured to determine physiological characteristics such as artery diameter and distension.

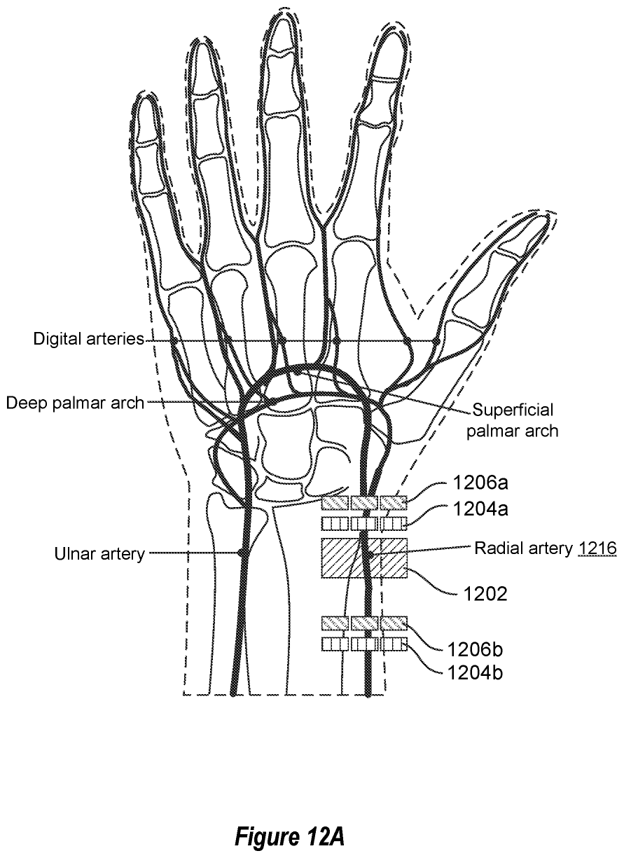

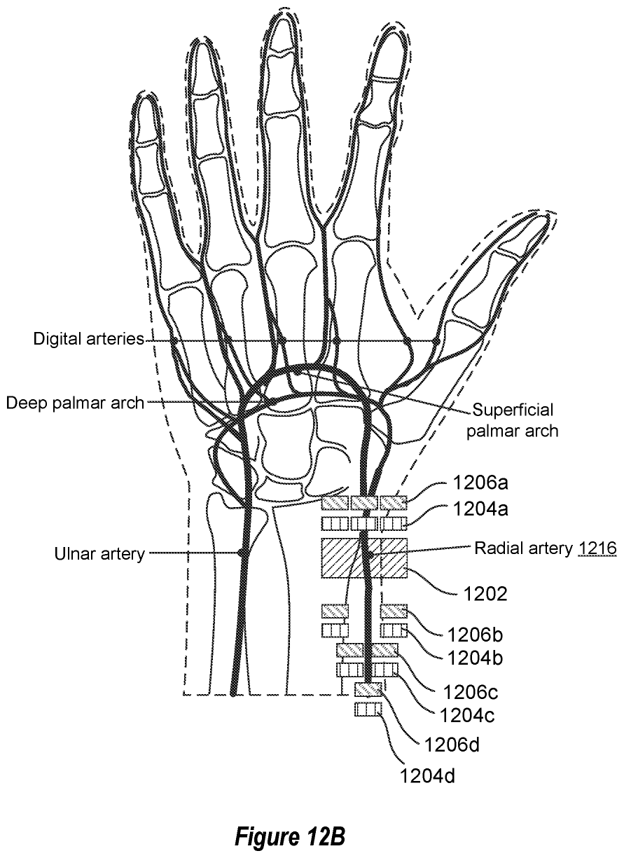

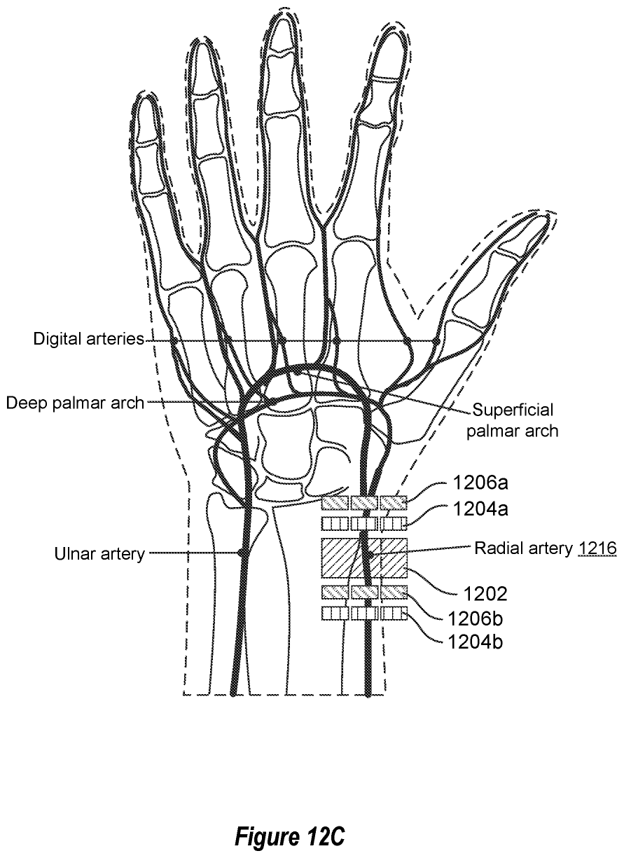

Multiple photoacoustic sensor systems 1101a-1101c may be implemented. As shown in FIG. 11A, these photoacoustic sensor systems 1101a-1101c are positioned orthogonally to the direction of the blood vessel 1116, which may provide area diversity to detect acoustic signals from or otherwise associated with the blood vessel 1116. However, it will be understood that myriad configurations, placements, and quantities (including one photoacoustic sensor system) of each of the components discussed with respect to FIGS. 11A and 11B are possible, especially in view of FIGS. 12A-12C.

FIG. 11B shows another view of the example configuration of a PAPG-based multi-sensor apparatus 1100, according to some embodiments. As opposed to FIG. 11A, this view shows a longitudinal section of the body part 1115 of the user where the photoacoustic sensor system 1101b, acoustic sensor systems 1102a, 1102b, and motion sensor systems 1103a, 1103b are located.

In some embodiments, the acoustic sensor systems 1102a and 1102b are spaced a first prescribed distance apart, at a first distance of ΔL₁. In some embodiments, the motion sensor systems 1103a and 1103b are spaced a second prescribed distance apart, at a second distance of ΔL₂. In some embodiments, the first distance and the second distance may be the same or substantially the same. That is, in such cases, ΔL₁ may be substantially equal to ΔL₂. However, ΔL₁ need not necessarily be equal to ΔL₂ and may be different, as long as those first and second distances will be known to the controller 808 and/or control system 706.

Each of acoustic sensor systems 1102a, 1102b may be an example of acoustic sensor system 702, and hence may be configured to detect acoustic signals (e.g., sound waves 1112) generated by pulses of pressure waves in the blood vessel 1116 and the motion of arterial walls. Since the acoustic sensor systems 1102a, 1102b are at two locations ΔL₁ apart, the time to of detecting acoustic signals by one acoustic sensor system 1102a and the time t₁ of detecting acoustic signals by another acoustic sensor system 1102b are different. This difference in time, Δt (equal to t₁−t₀), can be used to estimate a characteristic of the blood vessel 1116. More specifically, an arterial characteristic such as pulse wave velocity (PWV) may be determined based on acoustic signals with the following relationship between ΔL₁ and Δt:

$$PWV = Δ {L}_{1} / Δ t \qquad ( Eqn . 1 )$$

Each of motion sensor systems 1103a, 1103b may be an example of motion sensor system 703, and hence may be configured to detect motion, acceleration, or other inertial measurements based on motion (e.g., ΔD from distension or contraction as shown in FIG. 11B) or waves 1113 caused by the motion of blood vessel 1116 and tissue of the body part 1115. Since the motion sensor systems 1103a, 1103b are at two locations ΔL₂ apart, the time to of detecting motion signals by one motion sensor system 1103a and the time t₁ of detecting motion signals by another motion sensor system 1103b are different. PWV can be calculated based on motion signals with the following relationship between ΔL₂ and Δt:

$$PWV = Δ {L}_{2} / Δ t \qquad ( Eqn . 2 )$$

Assume for simplicity of illustration that ΔL₁ and ΔL₂ are equal in this example configuration and that first acoustic signals and motion signals are received at t₁ and second acoustic signals and motion signals are received at to. If ΔL₁ and ΔL₂ were different because the acoustic sensor systems 1102a, 1102b were placed apart at a difference distance than the motion sensor systems 1103a, 1103b were placed apart, PWV based on acoustic signals would be determined with a different value of Δt than that for PWV based on motion signals. As an illustrative example, if ΔL₂ were greater than ΔL₁, the time for distension or other motion of the blood vessel 1116 to propagate along the blood vessel 1116 would be longer, and thus, Δt₂ used to determine PWV based on motion signals would be greater than Δt₁ used to determine PWV based on acoustic signals.

To illustrate, measurements by a photoacoustic sensor system can be represented by waveform 1132 shown in FIG. 11C. Measurements by acoustic sensor systems or by motion sensor systems can be represented by waveforms 1142 taken by a first sensor and 1142′ taken by a second sensor, separated by Δt, shown in FIG. 11D.

Thus, the PAPG-based multi-sensor apparatus 1100 includes multiple points of measurement: (1) at least one photoacoustic sensor system 1101b that estimates artery diameter, distension, volumetric changes, and/or heart rate waveforms; (2) two or more acoustic sensor systems (e.g., microphones or microphone arrays) that detect small pressure changes caused by motion of blood vessel or tissue or skin at different locations; (3) two or more motion sensor systems (e.g., voice accelerometers or accelerometers or arrays thereof) that detect movement in the arterial wall caused by arterial distension and thus movement of the tissue or skin. Accelerometers can also be used to reject environmental noise and also detect larger movements.

In alternate embodiments, more than one photoacoustic sensor system could be placed along the blood vessel 1116. However, having one photoacoustic sensor system (such as only one photoacoustic sensor system 1101b) along the blood vessel 1116 would advantageously reduce costs, setting aside the photoacoustic sensor systems 1101a and 1101c placed for area diversity and coverage as illustrated in FIG. 11A.

Depending on configuration, at least a photoacoustic sensor system, a plurality of acoustic sensor systems, and a plurality of motion sensor systems can be arranged in different locations or positions relative to one another.

FIG. 12A shows an example layout of sensors of a PAPG-based multi-sensor apparatus having a photoacoustic sensor system 1202, acoustic sensor systems (including acoustic sensor systems 1204a, 1204b), and motion sensor systems (including motion sensor systems 1206a, 1206b). As can be seen, the photoacoustic sensor system 1202 may be disposed between a plurality of acoustic sensor systems (which includes acoustic sensor system 1204a) and another plurality of acoustic sensor systems (which includes acoustic sensor system 1204b). Similarly, the photoacoustic sensor system 1202 may be disposed between a plurality of motion sensor systems (which includes motion sensor system 1206a) and another plurality of motion sensor systems (which includes motion sensor system 1206b). In some configurations, the distance between each pair of acoustic sensor systems 1204a, 1204b may be substantially equal. In some configurations, the distance between each pair of motion sensor systems 1206a, 1206b may be substantially equal. In some configurations, the distance between the layers of acoustic sensor systems may be substantially equal to the distance between the layers of motion sensor systems. In some configurations, the aforementioned distances between layers and even individual sensors may be different.

FIG. 12B shows another example layout of sensors of a PAPG-based multi-sensor apparatus. There may be multiple layers or groups of acoustic sensor systems and motion sensor systems. For example, a first layer of acoustic sensor systems having three acoustic sensor systems including 1204a may be disposed adjacent to the photoacoustic sensor system 1202, a second layer of acoustic sensor systems having two acoustic sensor systems including 1204b may be disposed farther down a target object (e.g., blood vessel 1216), a third layer of acoustic sensor systems having two acoustic sensor systems including 1204c may be disposed adjacent to the second layer, and a fourth layer of acoustic sensor systems having one acoustic sensor system 1204d may be disposed adjacent to the third layer. Similarly, layers of motion sensor systems having motion sensor systems 1206a, 1206c, 1206c, 1206d, respectively, may be arranged at similar distances apart as the layers of acoustic sensor systems. In some configurations, the distance between acoustic sensor systems 1204a and 1204b may be substantially equal to the distance between motion sensor systems 1206a and 1206b. In some configurations, the distance between acoustic sensor systems 1204b and 1204c may be substantially equal to the distance between motion sensor systems 1206b and 1206c. In some configurations, the distance between acoustic sensor systems 1204c and 1204d may be substantially equal to the distance between motion sensor systems 1206d and 1206d. In some configurations, the aforementioned distances between layers and even individual sensors may be different.

FIG. 12C shows another example layout of sensors of a PAPG-based multi-sensor apparatus. The layout is similar to that of FIG. 12A, but the layer having acoustic sensor system 1204b and the layer having motion sensor system 1206b may be disposed closer to the photoacoustic sensor system 1202. Hence, the distance between the acoustic sensor systems 1204a and 1204b and the distance between the motion sensor systems 1206a and 1206b in FIG. 12B may be less than in FIG. 12A. As long as the distances are known, it will be possible to determine or estimate physiological characteristics or parameters such as PWV and blood pressure as described elsewhere herein.

The examples of FIGS. 12A-12C are merely illustrative and are not limiting. Literally limitless configurations and layouts are possible based on operational and design needs given the present disclosure.

Information from all three modalities can be used to determine arterial diameter and distension from the photoacoustic sensor system, and PWV from two locations of microphones and voice accelerometers, giving rise to a more accurate and complete picture of the physiological conditions and parameters of the user. Measurements from one or more modalities can corroborate and enhance measurements from other modalities. For instance, acoustic or motion sensing data can be useful complementary information to photoacoustic sensing data. As another non-limiting example, motion sensing signals may be weaker in some cases but may be confirmed by other modalities. Extraneous signals or interference signals from one modality may also be compensated with signals from another modality. In some implementations, blood pressure can be determined from the above information, as PWV is correlated to blood pressure as discussed above with respect to FIG. 5.

Furthermore, in some embodiments, a machine learning model (or more broadly an artificial intelligence model) may be used to predict a physiological parameter, e.g., blood pressure. A machine learning model may refer to a computational algorithm that indicates relationships between input variables and output variables. In some embodiments, a machine learning model can be trained. Training a machine learning model may involve, among other things, determining values of weights associated with the machine learning model, where relationships between the input variables and the output variables are based at least in part on the determined weight values. In one implementation, a machine learning model may be trained in a supervised manner using a training set that includes labeled training data. In a more particular example, the labeled training data may include inputs and manually annotated outputs that the machine learning model is to approximate using determined weight values. In another implementation, a machine learning model may be trained in an unsupervised manner in which weight values are determined without manually labeled training data.

An example training process for the artificial intelligence model or the machine learning model may involve providing training data that includes known photoacoustic signal data, known acoustic signal data, and known motion signal data, as well as the “ground truth” or the known output parameters, e.g., PTT, PWV, blood pressure, or cardiac features (e.g., peaks) that are known. In some approaches, a portion (e.g., 20%) of the training data may be used as part of a validation set for the machine learning model. With this training data and validation set, one or more loss functions may be implemented. A loss function is an optimization function in which an error is iteratively minimized through, e.g., gradient descent. A productive learning rate that dictates the “step” the gradient descent takes when finding the lowest error may be set during training as well.

As a result, a trained artificial intelligence model or a trained machine learning model can be generated. In some implementations, such a trained model can be used to further enhance the accuracy and reliability of the estimated physiological characteristics or parameter. For example, estimation derived from the obtained measurements from the three modalities can be provided to the machine learning model (stored at an apparatus and/or accessible by a control system thereof) to compare with a physiological characteristic of a blood vessel (e.g., PTT, PWV, heart rate) or a physiological parameter of a user (e.g., blood pressure) estimated by the machine learning model. If there is a discrepancy between the sensor-based measurements and the mode-generated prediction which is greater than a threshold, the obtained measurements may be further evaluated or discarded. If discarded, the model-generated estimation may be used, or additional measurements may be taken by the sensors. In some cases, measurements from a fallback modality may be selected (e.g., only the photoacoustic sensor system(s)) as a legacy approach rather than using multiple modalities (e.g., two or more modalities). On the other hand, if the discrepancy is lower than a threshold, the combined measurements or individual measurements from individual modalities may be selected or kept for further processing, reporting, displaying to the user, etc.

### Example Multi-Sensor PPG Apparatus

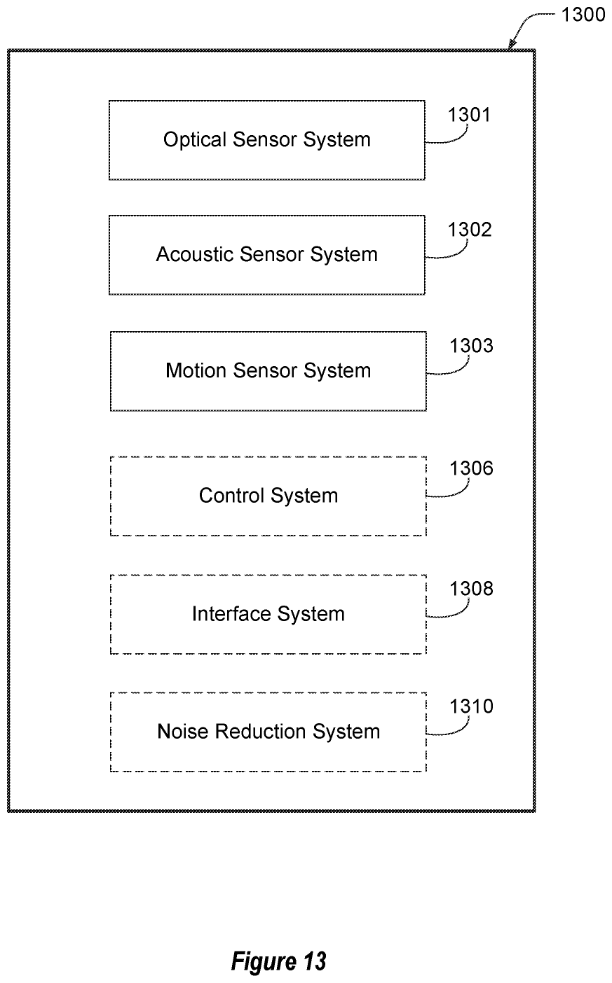

FIG. 13 is a block diagram that shows example components of a PPG-based multi-sensor apparatus according to some disclosed implementations. In this example, the apparatus 1300 may include an optical sensor system 1301, an acoustic sensor system 1302, and a motion sensor system 1303. Some implementations of the apparatus 1300 may include a control system 1306, an interface system 1308, a noise reduction system 1310, or combinations thereof.

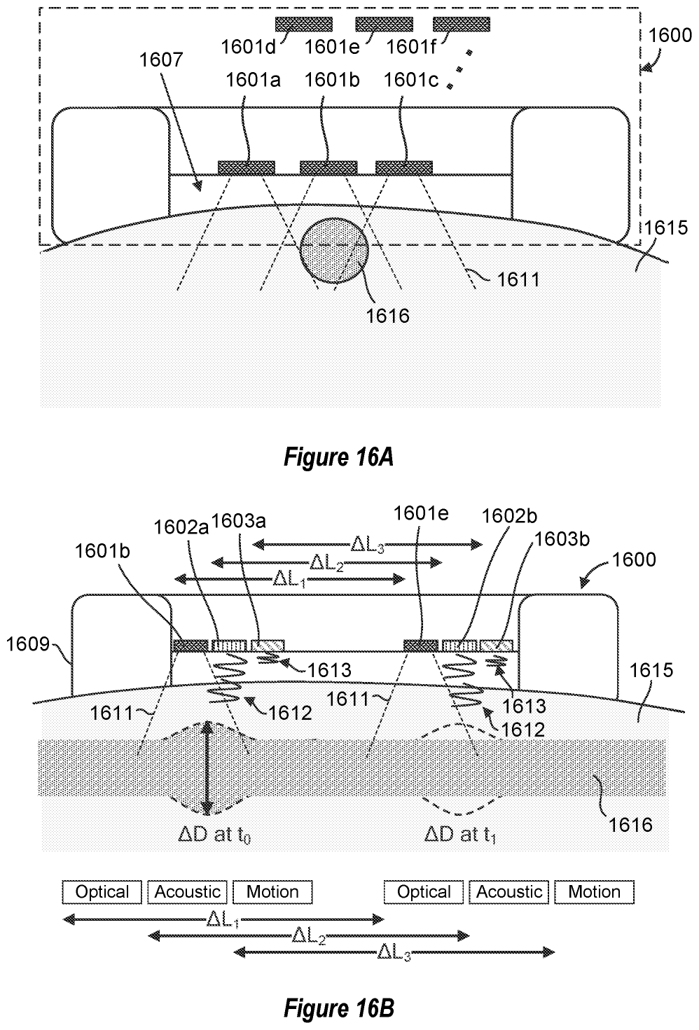

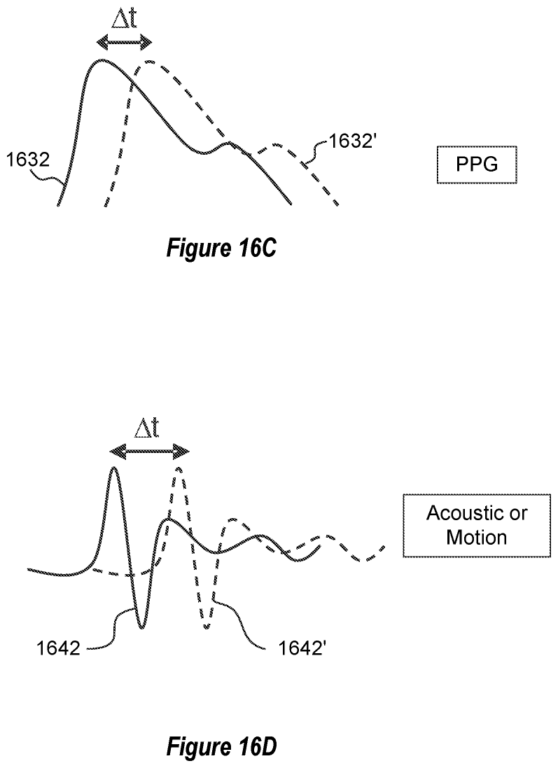

In some implementations, the interface system 1308 (including, e.g., a contact surface) may be included so as to allow contact with skin to maximize the sensitivity of the acoustic sensor system 1302 and/or the motion sensor system 1303. However, in some implementations, there may not be a contact surface. In such non-contact sensing implementations, measurement of signals from a target object (e.g., blood vessel) can be performed without applying pressure on the skin or the target object. Instead, an air gap may be present between the sensors and the skin and the target object in some implementations, as will be described further with respect to FIGS. 16A and 16B.

Various configurations of the optical sensor system 1301, acoustic sensor system 1302, and motion sensor system 1303 are disclosed herein. In some embodiments, optical sensor system 1301 may include a light source system and a receiver system (which may include at least one photodetector), and may be an example of the blood pressure monitoring device based on PPG as shown in FIG. 2. More specifically, part of the transmitted optical energy is absorbed in tissue and blood vessel, and part of it is reflected. Reflected optical signals can be detected by the optical sensor system 1301 (e.g., at the photodetector), which correlates inversely to absorption in blood and therefore inversely with volumetric flow of blood. Photodetectors may be configured to detect light in the visible spectrum, infrared spectrum, or both (such as that emitted by a light source or light source system). A photodiode may be an example of a photodetector.

In some implementations, the optical sensor system 1301 may also include a controller. Such controller may be communicatively coupled to the light source system configured to control the light source system to emit light toward a target object. The controller may also be communicatively coupled to a receiver system configured to detected optical signals from the target object. The controller may be configured to select at least one of a plurality of photodetectors. Such selected photodetectors may correspond to the best signals from multiple photodetectors. In some embodiments, the selection of the at least one photodetector may be based on information regarding detected optical signals. For example, signal quality or signal strength (based, e.g., on signal-to-noise ratio (SNR)) of some signals may be relatively higher than some others or above a prescribed threshold or percentile, which may indicate the best signals. In some implementations, the controller may also be configured to, based on the information regarding detected acoustic signals, determine or estimate at least one characteristic of the blood vessels such as PWV, arterial dimensions, or both.

In some embodiments, the acoustic sensor system 1302 may be an example of the acoustic sensor system 702. Some implementations of the apparatus 1300 may thus include one or more microphone elements similar to MEMS microphones 902a-902c shown and described with respect to FIG. 9. In some embodiments, the motion sensor system 1303 may be an example of the motion sensor system 703. Some implementations of the apparatus 1300 may thus include one or more accelerometers such as the spring-mass accelerometer 1000 shown and described with respect to FIG. 10 (or other types of motion sensors such as an inertial sensor, voice accelerometer, or a gyroscope).

Some implementations of the apparatus 1300 may include a control system 1306, an interface system 1308, a noise reduction system 1310, or combinations thereof.

The controller of the optical sensor system 1301 and/or the control system 1306 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The controller and/or the control system 1306 also may include (and/or be configured for communication with) one or more memory devices, such as one or more random access memory (RAM) devices, read-only memory (ROM) devices, etc. Accordingly, the optical sensor system 1301 may have a memory system that includes one or more memory devices. The controller and/or the control system 1306 may be configured for receiving and processing data from the receiver system, e.g., as described below. If the optical sensor system 1301 includes an ultrasonic transmitter, the controller and/or the control system 1306 may be configured for controlling the ultrasonic transmitter. In some implementations, functionality of the controller and/or the control system 1306 may be partitioned between one or more controllers or processors, such as a dedicated sensor controller and an applications processor of a mobile device.

In some examples, the control system 1306 may be communicatively coupled to the light source system of the optical sensor system 1301 and configured to control the light source system to emit light towards a target object. In further examples, the control system 1306 may be communicatively coupled to the receiver system of the optical sensor system 1301 and configured to detect acoustic signals from the target object. The control system 1306 may be configured to perform similar functionality as the controller of the optical sensor system 1301, e.g., with respect to the light source system and the receiver system of the optical sensor system 1301.

Some implementations of the apparatus 1300 may include an interface system 1308. The interface system 1308 may be an example of the interface system 708. Some implementations of the apparatus 1300 may include a noise reduction system 1310. The noise reduction system 1310 may be an example of the noise reduction system 710.

The apparatus 1300 may be used in a variety of different contexts, many examples of which are disclosed herein. For example, in some implementations a mobile device may include the optical sensor system 1301, the acoustic sensor system 1302, and the motion sensor system 1303. In some such examples, the mobile device may be a smart phone. In some implementations, a wearable device may include the photoacoustic sensor system 1301, the acoustic sensor system 1302, and the motion sensor system 1303. The wearable device may, for example, be a bracelet, an armband, a wristband, a watch, a ring, a headband or a patch. In certain embodiments, the wearable device may be one or a pair of earbuds, a headset, a head rest mount, a headband, headphones, or another head-wearable or head-mounted device.

### Example Configurations of PAPG Apparatus

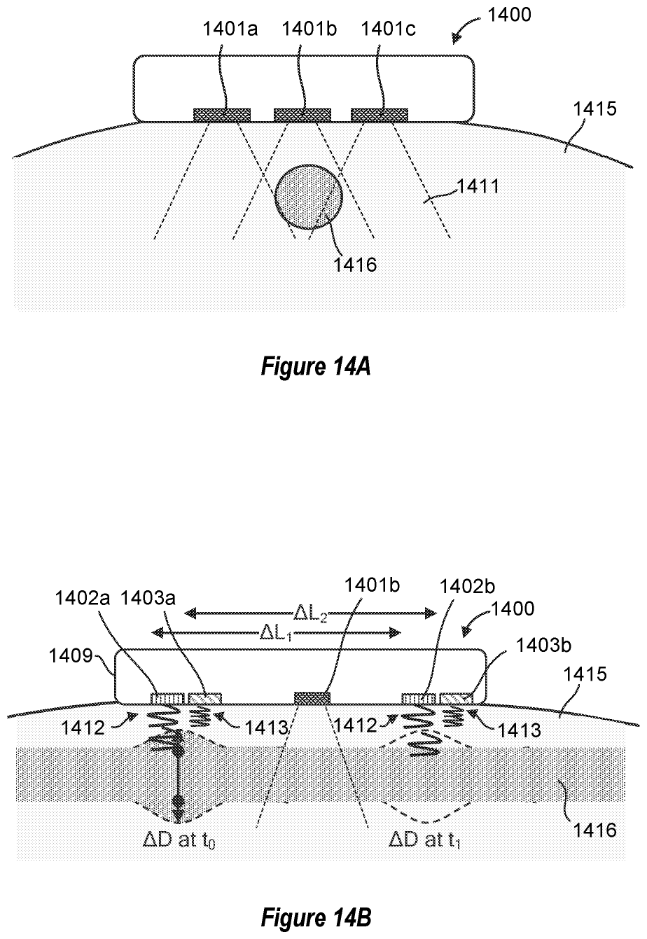

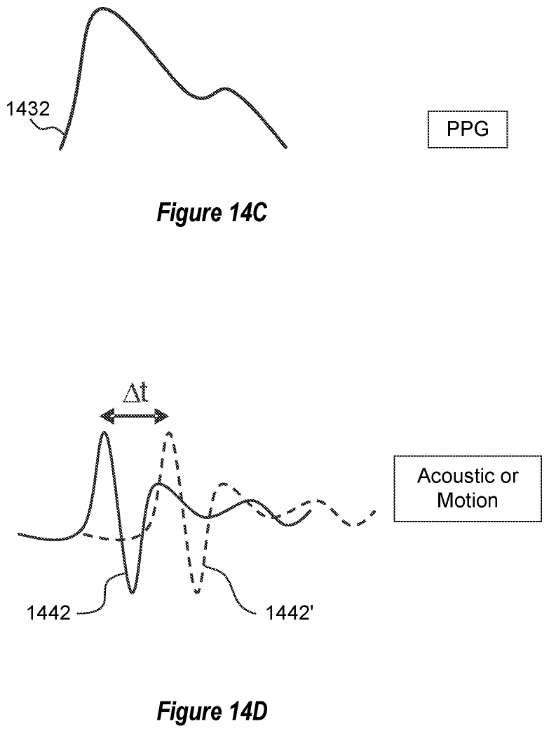

FIG. 14A shows one view of an example configuration of a PPG-based multi-sensor apparatus 1400, according to some embodiments. The PPG-based multi-sensor apparatus 1400 may be an example of apparatus 1300, and may have components including one or more optical sensor systems 1401a-1401c, a plurality of acoustic sensor systems 1402a, 1402b, and a plurality of motion sensor systems 1403a, 1403b. This view shows a cross-sectional view of a body part 1415 of a user (e.g., finger 115) where the optical sensor systems 1401a-1401c are located. In some implementations (as shown in FIG. 14B), the foregoing components may abut a surface of a housing structure 1409, where the surface is contactable with the body part 1415 of the user (e.g., via a contact surface of an interface system 1308) to maximize sensitivity.

Each of optical sensor systems 1401a-1401c may be an example of optical sensor system 1301, and hence may be configured to emit optical signals (e.g., light 1411) and detect optical signals that result from a target object or tissue of interest, such as blood vessel 1416 of the body part 1415 of the user, according to principles described with respect to FIG. 2. Each optical sensor system may be configured to determine physiological characteristics of the blood vessel such as volumetric blood flow—e.g., over time using multiple optical sensor systems spaced apart along the blood vessel. Volumetric flow corresponds to arterial distention. Another example of a physiological characteristic of the blood vessel that can be captured using optical measurements is blood oxygen level.

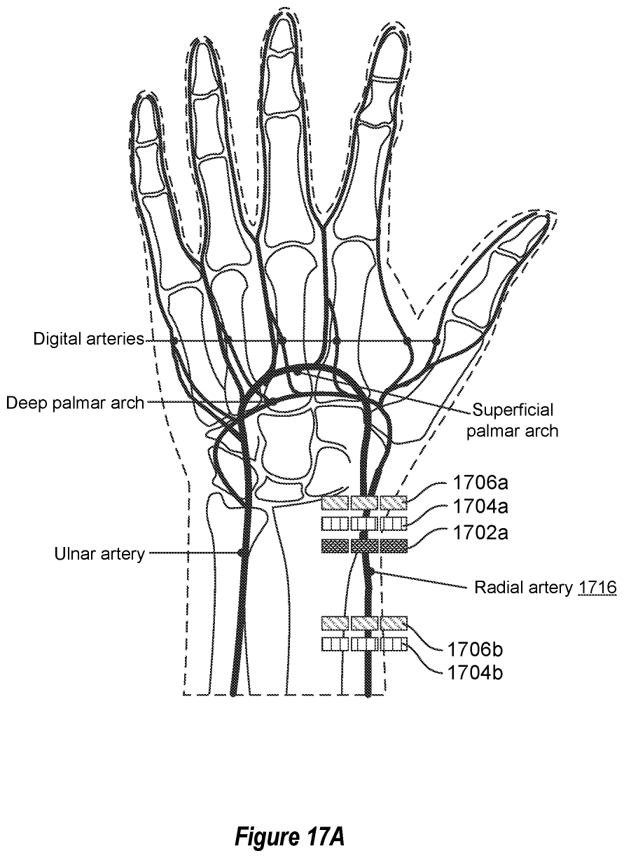

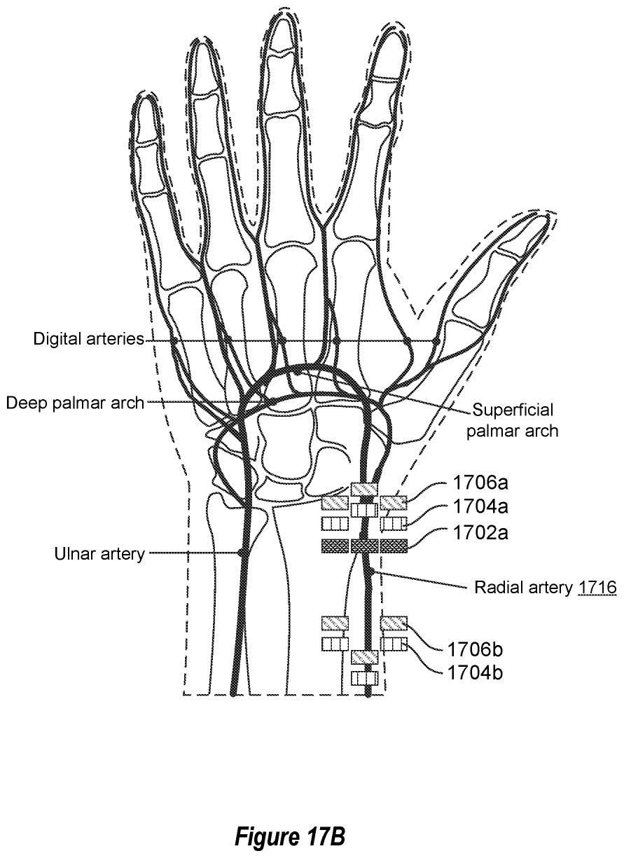

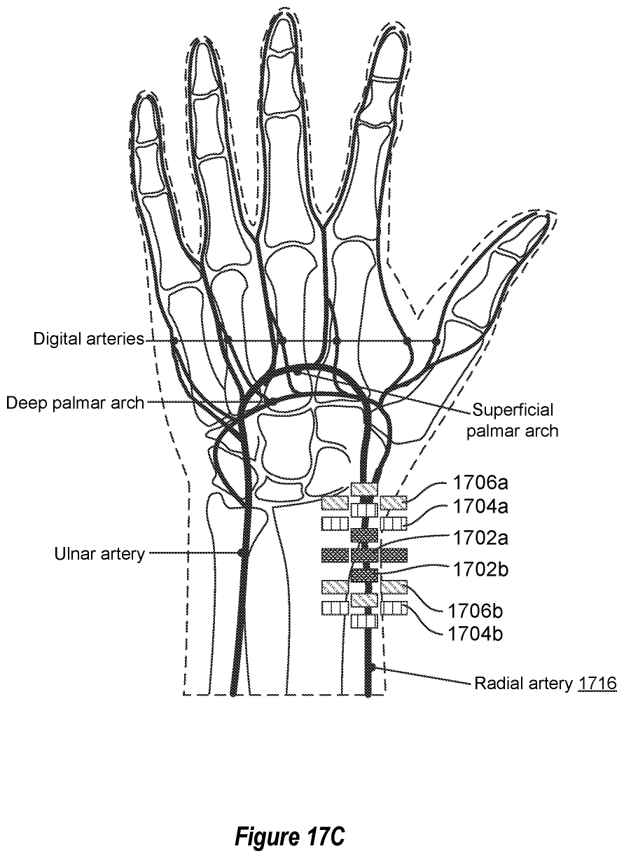

Multiple optical sensor systems 1401a-1401c may be implemented. As shown in FIG. 14A, these optical sensor systems 1401a-1401c are positioned orthogonally to the direction of the blood vessel 1416, which may provide area diversity to detect optical signals from or otherwise associated with the blood vessel 1416. However, it will be understood that myriad configurations, placements, and quantities (including one optical sensor system) of each of the components discussed with respect to FIGS. 14A and 14B are possible, especially in view of FIGS. 17A-17C.

FIG. 14B shows another view of the example configuration of a PPG-based multi-sensor apparatus 1400, according to some embodiments. As opposed to FIG. 14A, this view shows a longitudinal section of the body part 1415 of the user where the optical sensor system 1401b, acoustic sensor systems 1402a, 1402b, and motion sensor systems 1403a, 1403b are located.

In some embodiments, the acoustic sensor systems 1402a and 1402b are spaced a first prescribed distance apart, at a first distance of ΔL₁. In some embodiments, the motion sensor systems 1403a and 1403b are spaced a second prescribed distance apart, at a second distance of ΔL₂. In some embodiments, the first distance and the second distance may be the same or substantially the same. That is, in such cases, ΔL₁ may be substantially equal to ΔL₂. However, ΔL₁ need not necessarily be equal to ΔL₂ and may be different, as long as those first and second distances will be known to the controller of the optical sensor system 1401b and/or control system 1306.

Each of acoustic sensor systems 1402a, 1402b may be an example of acoustic sensor system 1302, and hence may be configured to detect acoustic signals (e.g., sound waves 1412) generated by pulses of pressure waves in the blood vessel 1416 and the motion of arterial walls. Since the acoustic sensor systems 1402a, 1402b are at two locations ΔL₁ apart, the time to of detecting acoustic signals by one acoustic sensor system 1402a and the time t₁ of detecting acoustic signals by another acoustic sensor system 1402b are different. In some approaches, an arterial characteristic such as PWV may be determined based on acoustic signals based on the relationship between ΔL₁ and Δt (equal to t₁−t₀), e.g., using Eqn. 1 or 2.

Each of motion sensor systems 1403a, 1403b may be an example of motion sensor system 1303, and hence may be configured to detect motion, acceleration, or other inertial measurements based on motion (e.g., ΔD from distension or contraction as shown in FIG. 14B) or waves 1413 caused by the motion of blood vessel 1416 and tissue of the body part 1415. Since the motion sensor systems 1403a, 1403b are at two locations ΔL₂ apart, the time to of detecting motion signals by one motion sensor system 1403a and the time t₁ of detecting motion signals by another motion sensor system 1403b are different. PWV can be calculated based on motion signals with the following relationship between ΔL₂ and Δt, e.g., using Eqn. 1 or 2.

To illustrate, measurements by an optical sensor system can be represented by waveform 1432 shown in FIG. 14C. Measurements by acoustic sensor systems or by motion sensor systems can be represented by waveforms 1442 taken by a first sensor and 1442′ taken by a second sensor, separated by Δt, shown in FIG. 14D.

Thus, the PPG-based multi-sensor apparatus 1400 includes multiple points of measurement: (1) at least one optical sensor system 1401b that estimates volumetric blood flow; (2) two or more acoustic sensor systems (e.g., microphones or microphone arrays) that detect small pressure changes caused by motion of blood vessel or tissue or skin at different locations; (3) two or more motion sensor systems (e.g., voice accelerometers or accelerometers or arrays thereof) that detect movement in the arterial wall caused by arterial distension and thus movement of the tissue or skin. Accelerometers can also be used to reject environmental noise and also detect larger movements.

In some embodiments, more than one optical sensor system could be placed along the blood vessel 1416. Not only would using multiple optical sensor systems increase area diversity and coverage, but it would enable further extraction of information (e.g., PWV), as will now be discussed.

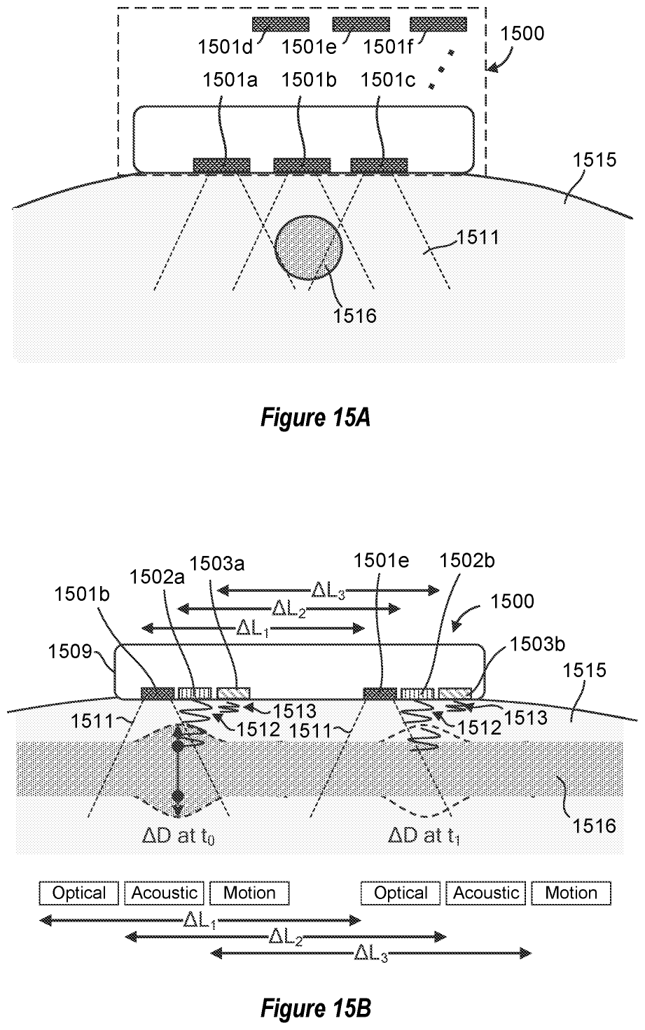

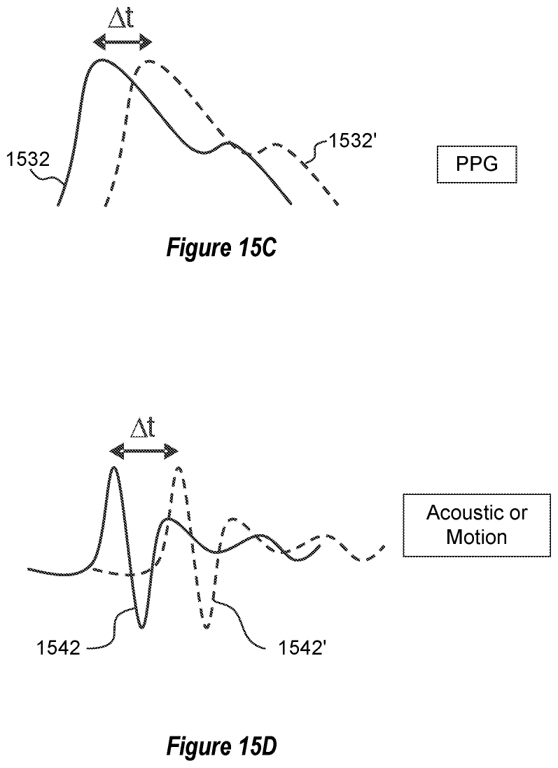

FIG. 15A shows one view of another example configuration of a PPG-based multi-sensor apparatus 1500, according to some embodiments. Apparatus 1500 may be an example of apparatus 1300. One or more optical sensor systems 1501a-1501f may each be an example of optical sensor system 1301, and hence may be configured to emit optical signals (e.g., light 1511) and detect optical signals that result from a target object or tissue of interest, such as blood vessel 1516 of the body part 1515 of the user, according to principles described with respect to FIG. 2.

Note that, in this example apparatus 1500, more than one layer or group of optical sensor systems may be used, such as a first layer with optical sensor systems 1501a-1501c and a second layer with optical sensor systems 1501d-1501f. Optical sensor systems 1501d-1501f may be disposed in a location that extends into and out of the illustration. In some configurations, a layer may be disposed substantially orthogonally to the direction of the blood vessel 1516 for area diversity for detection of optical signals. In some configurations, a line that extends through optical sensor systems 1501a-1501c may be substantially parallel to a line that extends through optical sensor systems 1501d-1501f. However, various placements and quantities of optical sensor systems per layer or group are possible, as will become more evident in view of FIG. 17C.

Each optical sensor system may be configured to determine physiological characteristics of the blood vessel such as volumetric blood flow—e.g., over time using multiple optical sensor systems spaced apart along the blood vessel. In addition, by using two (or more) optical sensor systems spaced apart at a prescribed distance (e.g., distance of ΔL₁), PWV may be calculated using the known distance ΔL₁ and the time difference of measurements Δt (equal to t₁−t₀), e.g., using Eqn. 1 or 2.

FIG. 15B shows another view of the FIG. 15A example configuration of a PPG-based multi-sensor apparatus 1500, according to some embodiments. As opposed to FIG. 15A, this views shows a longitudinal section of the body part 1515 of the user where optical sensor systems 1501b, 1501e, acoustic sensor systems 1502a, 1502b, and motion sensor systems 1503a, 1503b are located.

In some embodiments, the optical sensor systems 1501b and 1501e are spaced a first prescribed distance apart, at a first distance of ΔL₁. In some embodiments, motion sensor systems 1502a and 1502b are spaced a second prescribed distance apart, at a second distance of ΔL₂. In some embodiments, the motion sensor systems 1503a and 1503b are spaced a third prescribed distance apart, at a third distance of ΔL₃. In some configurations, two or more of the first distance, the second distance, or the third distance may be the same or substantially the same. However, they may all be different in other configurations.

In some implementations (as shown in FIG. 15B), the foregoing sensor systems may abut a surface of a housing structure 1509, where the surface is contactable with the body part 1515 of the user (e.g., via a contact surface of an interface system 1308) to maximize sensitivity.

Each of acoustic sensor systems 1502a, 1502b may be an example of acoustic sensor system 1302, and hence may be configured to detect acoustic signals (e.g., sound waves 1512) generated by pulses of pressure waves in the blood vessel 1516 and the motion of arterial walls. Since the acoustic sensor systems 1502a, 1502b are at two locations ΔL₁ apart, the time to of detecting acoustic signals by one acoustic sensor system 1502a and the time t₁ of detecting acoustic signals by another acoustic sensor system 1502b are different. In some approaches, an arterial characteristic such as PWV may be determined based on acoustic signals based on the relationship between ΔL₂ and Δt (equal to t₁−t₀), e.g., using Eqn. 1 or 2.

Each of motion sensor systems 1503a, 1503b may be an example of motion sensor system 1303, and hence may be configured to detect motion, acceleration, or other inertial measurements based on motion (e.g., ΔD from distension or contraction as shown in FIG. 15B) or waves 1513 caused by the motion of blood vessel 1516 and tissue of the body part 1515. Since the motion sensor systems 1503a, 1503b are at two locations ΔL₂ apart, the time to of detecting motion signals by one motion sensor system 1503a and the time t₁ of detecting motion signals by another motion sensor system 1503b are different. PWV can be calculated based on motion signals with the following relationship between ΔL₃ and Δt, e.g., using Eqn. 1 or 2.

To illustrate, measurements by optical sensor systems can be represented by waveforms 1532 and 1532′ shown in FIG. 15C. For example, waveform 1532 may correspond to optical sensor system 1501b, and waveform 1532′ may correspond to optical sensor system 1501e, separated by Δt. Measurements by acoustic sensor systems or by motion sensor systems can be represented by waveforms 1542 taken by a first sensor and 1542′ taken by a second sensor, separated by Δt, shown in FIG. 15D.

Thus, the PPG-based multi-sensor apparatus 1500 includes multiple points of measurement: (1) two or more optical sensor systems 1501b, 1501e that estimate volumetric blood flow (and/or PWV); (2) two or more acoustic sensor systems (e.g., microphones or microphone arrays) that detect small pressure changes caused by motion of blood vessel or tissue or skin at different locations; (3) two or more motion sensor systems (e.g., voice accelerometers or accelerometers or arrays thereof) that detect movement in the arterial wall caused by arterial distension and thus movement of the tissue or skin. Accelerometers can also be used to reject environmental noise and also detect larger movements.

FIG. 16A shows one view of another example configuration of a PPG-based multi-sensor apparatus 1600, according to some embodiments. Apparatus 1600 may be an example of apparatus 1300. Similar to FIG. 15A, one or more optical sensor systems 1601a-1601f may each be an example of optical sensor system 1301, and hence may be configured to emit optical signals (e.g., light 1611) and detect optical signals that result from a target object or tissue of interest, such as blood vessel 1616 of the body part 1615 of the user, according to principles described with respect to FIG. 2.

Each optical sensor system may be configured to determine physiological characteristics of the blood vessel such as volumetric blood flow—e.g., over time using multiple optical sensor systems spaced apart along the blood vessel. In addition, by using two (or more) optical sensor systems spaced apart at a prescribed distance (e.g., distance of ΔL₁), PWV may be calculated using the known distance ΔL₁ and the time difference of measurements Δt (equal to t₁−t₀), e.g., using Eqn. 1 or 2.

FIG. 16B shows another view of the FIG. 16A example configuration of a PPG-based multi-sensor apparatus 1600, according to some embodiments. As opposed to FIG. 16A, this views shows a longitudinal section of the body part 1615 of the user where optical sensor systems 1601b, 1601e, acoustic sensor systems 1602a, 1602b, and motion sensor systems 1603a, 1603b are located.

In some embodiments, the optical sensor systems 1601b and 1601e are spaced a first prescribed distance apart, at a first distance of ΔL₁. In some embodiments, motion sensor systems 1602a and 1602b are spaced a second prescribed distance apart, at a second distance of ΔL₂. In some embodiments, the motion sensor systems 1603a and 1603b are spaced a third prescribed distance apart, at a third distance of ΔL₃. In some configurations, two or more of the first distance, the second distance, or the third distance may be the same or substantially the same. However, they may all be different in other configurations.

Each of acoustic sensor systems 1602a, 1602b may be an example of acoustic sensor system 1302, and hence may be configured to detect acoustic signals (e.g., sound waves 1612) generated by pulses of pressure waves in the blood vessel 1616 and the motion of arterial walls. Since the acoustic sensor systems 1602a, 1602b are at two locations ΔL₁ apart, the time to of detecting acoustic signals by one acoustic sensor system 1602a and the time t₁ of detecting acoustic signals by another acoustic sensor system 1602b are different. In some approaches, an arterial characteristic such as PWV may be determined based on acoustic signals based on the relationship between ΔL₂ and Δt (equal to t₁−t₀), e.g., using Eqn. 1 or 2.

Each of motion sensor systems 1603a, 1603b may be an example of motion sensor system 1303, and hence may be configured to detect motion, acceleration, or other inertial measurements based on motion (e.g., ΔD from distension or contraction as shown in FIG. 16B) or waves 1613 caused by the motion of blood vessel 1616 and tissue of the body part 1615. Since the motion sensor systems 1603a, 1603b are at two locations ΔL₂ apart, the time to of detecting motion signals by one motion sensor system 1603a and the time t₁ of detecting motion signals by another motion sensor system 1603b are different. PWV can be calculated based on motion signals with the following relationship between ΔL₃ and Δt, e.g., using Eqn. 1 or 2.

In some embodiments, as shown in FIGS. 16A and 16B, the example apparatus 1600 may include an air gap 1607 between the aforementioned sensor systems and the body part 1615 of the user. The air gap 1607 allows measurement of signals from a target object (e.g., blood vessel 1616) without applying external pressure on the body part 1615 or the skin. For example, arterial signals can be measured without pressure on the artery. By not having areas of the skin being directly touched by a sensor, any extraneous or environmental pressure or force is removed. This can result in measurements that are not affected by external pressure because it is possible that such external pressure changes the diameter of the blood vessel. Measurements of the diameter after it has changed may affect measurements relating to the diameter, blood pressure, PWV, blood flow, motion, etc. Hence, the cavity created by the air gap 1607 can ensure there is no external pressure applied to the skin, at least in the sensing region. In these embodiments, the aforementioned sensor systems may abut a surface of a housing structure 1609. However, the surface is not contactable with the body part 1615 of the user. Instead of maximizing sensitivity, a configuration that includes the air gap 1607 can increase accuracy of what is occurring in the target object (e.g., blood vessel 1616).

Measurements by optical sensor systems can be represented by waveforms 1632 and 1632′ shown in FIG. 16C. For example, waveform 1632 may correspond to optical sensor system 1601b, and waveform 1632′ may correspond to optical sensor system 1601e, separated by Δt. Measurements by acoustic sensor systems or by motion sensor systems can be represented by waveforms 1642 taken by a first sensor and 1642′ taken by a second sensor, separated by Δt, shown in FIG. 16D.

Thus, the PPG-based multi-sensor apparatus 1600 includes multiple points of measurement: (1) two or more optical sensor systems 1601b, 1601e that estimate volumetric blood flow (and/or PWV); (2) two or more acoustic sensor systems (e.g., microphones or microphone arrays) that detect small pressure changes caused by motion of blood vessel or tissue or skin at different locations; (3) two or more motion sensor systems (e.g., voice accelerometers or accelerometers or arrays thereof) that detect movement in the arterial wall caused by arterial distension and thus movement of the tissue or skin. Accelerometers can also be used to reject environmental noise and also detect larger movements.

Depending on configuration, at least one or more optical sensor systems, a plurality of acoustic sensor systems, and a plurality of motion sensor systems can be arranged in different locations or positions relative to one another.

FIG. 17A shows an example layout of sensors of a PPG-based multi-sensor apparatus having an optical sensor system 1702a, acoustic sensor systems (including acoustic sensor systems 1704a, 1704b), and motion sensor systems (including motion sensor systems 1706a, 1706b). These sensor systems may be disposed along a target object (e.g., blood vessel 1716) in various groupings and distances from one another. In some configurations, the distance between the layers of acoustic sensor systems may be substantially equal to the distance between the layers of motion sensor systems. In some configurations, the aforementioned distances between layers and even individual sensors may be different.

FIG. 17B shows another example layout of sensors of a PPG-based multi-sensor apparatus having an optical sensor system 1702a, acoustic sensor systems (including acoustic sensor systems 1704a, 1704b), and motion sensor systems (including motion sensor systems 1706a, 1706b). There may be multiple layers or groups of acoustic sensor systems and motion sensor systems. Again, the sensor systems may be disposed in various groupings and distances from one another.

FIG. 17C shows another example layout of sensors of a PPG-based multi-sensor apparatus. Here, there may be multiple layers or groups of optical sensor systems, e.g., 1702a and 1702b. In this configuration, optical sensor systems 1702a and 1702b may be separated by a first prescribed distance (e.g., ΔL₁), acoustic sensor systems 1704a and 1704b may be separated by a second prescribed distance (e.g., ΔL₂), and motion sensor systems 1706a and 1706b may be separated by a third prescribed distance (e.g., ΔL₃). In some configurations, two or more of the first distance, the second distance, or the third distance may be the same or substantially the same. For example, ΔL₂ may be substantially the same as ΔL₃, but ΔL₁ may be different from ΔL₂ and ΔL₃. They may all be the same or different in other configurations.

The examples of FIGS. 17A-17C are merely illustrative and are not limiting. Literally limitless configurations and layouts are possible based on operational and design needs given the present disclosure.

In the example configurations of FIGS. 14A, 14B, 15A, 15B, 16A and 16B, information from all three modalities can be used to determine volumetric blood flow (and/or PWV) from one or more optical sensor systems, and PWV from two locations of microphones and voice accelerometers, giving rise to a more accurate and complete picture of the physiological conditions and parameters of the user. Measurements from one or more modalities can corroborate and enhance measurements from other modalities. For instance, acoustic or motion sensing data can be useful complementary information to photoacoustic sensing data. As another non-limiting example, motion sensing signals may be weaker in some cases but may be confirmed by other modalities. Extraneous signals or interference signals from one modality may also be compensated with signals from another modality. In some implementations, blood pressure can be determined from the above information, as PWV is correlated to blood pressure as discussed above with respect to FIG. 5.

Furthermore, in some embodiments, a machine learning model may be used to predict a physiological parameter, e.g., blood pressure. A trained machine learning model can be generated from a training process as described above, and the trained machine learning model can be implemented to further enhance, complement, verify, or corroborate the sensor-obtained measurements.

In yet other embodiments, further modalities or modalities using different settings can be used in conjunction with the photoacoustic, optical, acoustic, and motion sensors described herein to further add to, enhance, or enable corroboration of measurement data obtained. As one example, emitted light from different photoacoustic or optical sensor systems can be in different wavelengths. As another example, ultrasound waves and sensors can be used for vascular measurements. As another example, radio frequency (RF) waves and sensors can be used to image a target object with RF representations.

In certain configurations, PAPG and PPG sensor systems may be used in conjunction in the same multi-sensor device. In some examples, at least one photoacoustic sensor system and at least one optical sensor system may be used together, each implemented according to the present disclosure, along with acoustic sensor systems and/or motion sensor systems also implemented according to the present disclosure. Photoacoustic and/or optical signal measurements may thus compensate for acoustic signals and/or motion signals, or vice versa. In these example configurations, four modalities may be deployed in the device.

### Example Methods

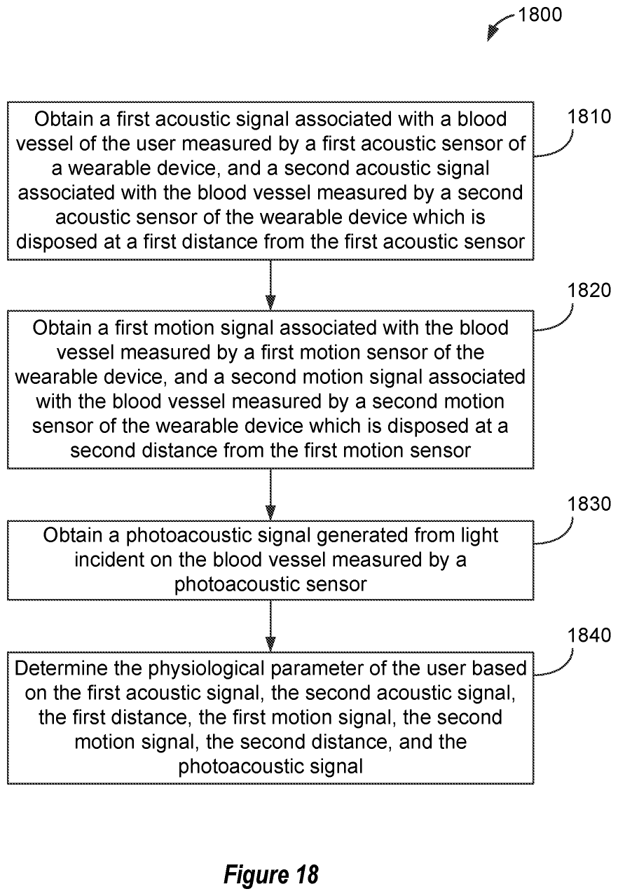

FIG. 18 is a flow diagram of a method 1800 for determining a physiological parameter of a user, according to some disclosed implementations. Structure for performing the functionality illustrated in one or more of the blocks shown in FIG. 18 may be performed by hardware and/or software components of a computerized apparatus or system (which may be implemented as a wearable device in some embodiments). Components of such apparatus or system may include, for example, one or more sensors, one or more light sources, one or more receiver elements, one or more optical waveguides, one or more control systems (including one or more processors), one or more memory, and/or one or more computer-readable apparatus including a storage medium storing computer-readable and/or computer-executable instructions that are configured to, when executed by the control system, cause the control system, one or more processors, or the apparatus to perform operations represented by blocks below. Example components of the apparatus are illustrated in FIGS. 7, 11A and 11B, which are described in more detail above.

The blocks of FIG. 18 may, for example, be performed by the apparatus 700 or apparatus 1100 or by a similar apparatus, or a component thereof (e.g., a control system). As with other methods disclosed herein, the method outlined in FIG. 18 may include more or fewer blocks than indicated. Moreover, the blocks of methods disclosed herein are not necessarily performed in the order indicated. In some instances, one or more of the blocks shown in FIG. 18 may be performed concurrently.

At block 1810, the method 1800 may include obtaining a first acoustic signal associated with a blood vessel of the user measured by a first acoustic sensor of a wearable device, and a second acoustic signal associated with the blood vessel measured by a second acoustic sensor of the wearable device which is disposed at a first distance from the first acoustic sensor.

Means for performing functionality at block 1810 may include an acoustic sensor system 702 and/or other components of the apparatus as shown in FIG. 7.

At block 1820, the method 1800 may include obtaining a first motion signal associated with the blood vessel measured by a first motion sensor of the wearable device, and a second motion signal associated with the blood vessel measured by a second motion sensor of the wearable device which is disposed at a second distance from the first motion sensor.

In some embodiments, the first and second acoustic sensors may each include a microphone, and the first and second motion sensors may each include an accelerometer. In some embodiments, the first acoustic sensor and the second acoustic sensor may be examples of the acoustic sensor system 702 or a component thereof. In some embodiments, the first acoustic sensor and the second acoustic sensor may be examples of microphone 902. In some embodiments, the first motion sensor and the second motion sensor may be examples of the motion sensor system 703 or a component thereof. In some embodiments, the first motion sensor and the second motion sensor may be examples of accelerometer 1000 (e.g., MEMS voice accelerometer) or another inertial sensor (e.g., gyroscope).

Means for performing functionality at block 1820 may include a motion sensor system 703 and/or other components of the apparatus as shown in FIG. 7.

At block 1830, the method 1800 may include obtaining a photoacoustic signal generated from light incident on the blood vessel measured by a photoacoustic sensor. In some embodiments, the first acoustic sensor, the second acoustic sensor, the first motion sensor, the second motion sensor, and the photoacoustic sensor may be contactable with a skin of the user. In some embodiments, the photoacoustic sensor may be an example of the photoacoustic sensor system 701 or a component thereof.

In some embodiments, the method 1800 may further include determining one or more physiological characteristics of the blood vessel based on the photoacoustic signal; wherein the one or more physiological characteristics may include a diameter of the blood vessel, volumetric blood flow, a distension of the blood vessel, or a combination thereof.

Means for performing functionality at block 1830 may include a photoacoustic sensor system 701 and/or other components of the apparatus as shown in FIG. 7.

At block 1840, the method 1800 may include determining the physiological parameter of the user based on the first acoustic signal, the second acoustic signal, the first distance, the first motion signal, the second motion signal, the second distance, and the photoacoustic signal.

In some embodiments, the method 1800 may further include determining a physiological characteristic of the blood vessel based on: the first distance and a first temporal difference, the first temporal difference comprising a difference in a measurement time of the first acoustic signal and a measurement time of the second acoustic signal; the second distance and a second temporal difference, the second temporal difference comprising a difference in measurement time of the first motion signal and a measurement time of the second motion signal; or a combination thereof. In some implementations, the physiological characteristic of the blood vessel may include a pulse wave velocity of the blood vessel. In some implementations, the determining of the physiological parameter of the user may include determining a blood pressure of the user; and the determining of the blood pressure of the user may be based on the pulse wave velocity of the blood vessel.

In some embodiments, the method 1800 may further include using a trained machine learning model configured to: receive the first acoustic signal, the second acoustic signal, the first motion signal, the second motion signal, the photoacoustic signal, or a combination thereof; and output a prediction the physiological parameter of the user.

In some embodiments, the method 1800 may further include waking up the first motion sensor, the second motion sensor, or the photoacoustic sensor from a low-power state responsive to the first acoustic sensor or the second acoustic sensor detecting an acoustic signal having a signal quality above a threshold.

Means for performing functionality at block 1840 may include a control system 706 and/or other components of an apparatus as shown in FIG. 7.

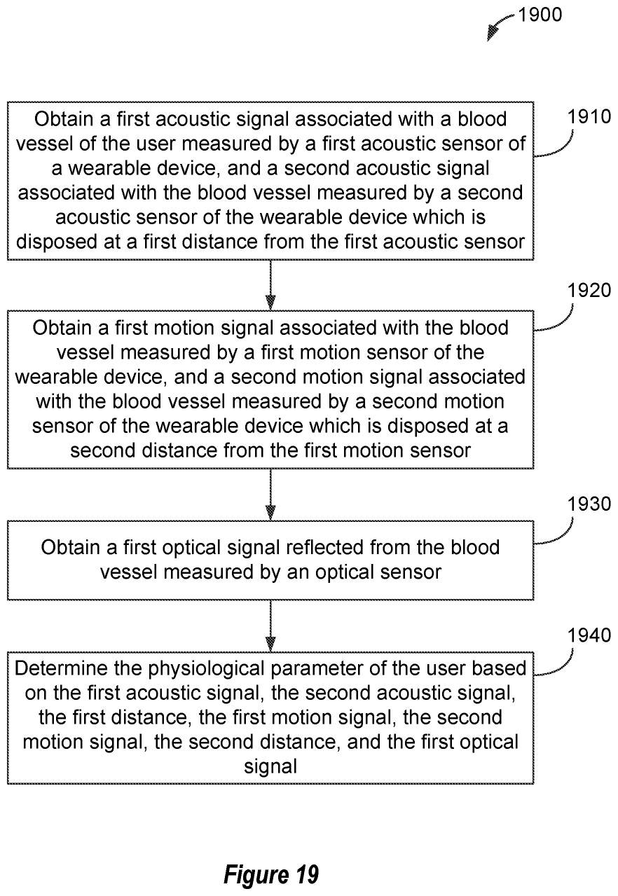

FIG. 19 is a flow diagram of another method 1900 for determining a physiological parameter of a user, according to some disclosed implementations. Structure for performing the functionality illustrated in one or more of the blocks shown in FIG. 19 may be performed by hardware and/or software components of a computerized apparatus or system (which may be implemented as a wearable device in some embodiments). Components of such apparatus or system may include, for example, one or more sensors, one or more light sources, one or more receiver elements, one or more optical waveguides, one or more control systems (including one or more processors), one or more memory, and/or one or more computer-readable apparatus including a storage medium storing computer-readable and/or computer-executable instructions that are configured to, when executed by the control system, cause the control system, one or more processors, or the apparatus to perform operations represented by blocks below. Example components of the apparatus are illustrated in FIGS. 13, 14A, 14B, 15A, 15B, 16A and 16B, which are described in more detail above.

The blocks of FIG. 19 may, for example, be performed by the apparatus 1300, 1400, 1500 or 1600 or by a similar apparatus, or a component thereof (e.g., a control system). As with other methods disclosed herein, the method outlined in FIG. 19 may include more or fewer blocks than indicated. Moreover, the blocks of methods disclosed herein are not necessarily performed in the order indicated. In some instances, one or more of the blocks shown in FIG. 19 may be performed concurrently.

At block 1910, the method 1900 may include obtaining a first acoustic signal associated with a blood vessel of the user measured by a first acoustic sensor of a wearable device, and a second acoustic signal associated with the blood vessel measured by a second acoustic sensor of the wearable device which is disposed at a first distance from the first acoustic sensor.

Means for performing functionality at block 1910 may include an acoustic sensor system 1302 and/or other components of the apparatus as shown in FIG. 13.

At block 1920, the method 1900 may include obtaining a first motion signal associated with the blood vessel measured by a first motion sensor of the wearable device, and a second motion signal associated with the blood vessel measured by a second motion sensor of the wearable device which is disposed at a second distance from the first motion sensor.

In some embodiments, the first and second acoustic sensors may each include a microphone, and the first and second motion sensors may each include an accelerometer. In some embodiments, the first acoustic sensor and the second acoustic sensor may be examples of the acoustic sensor system 1302 or a component thereof. In some embodiments, the first acoustic sensor and the second acoustic sensor may be examples of microphone 902. In some embodiments, the first motion sensor and the second motion sensor may be examples of the motion sensor system 1303 or a component thereof. In some embodiments, the first motion sensor and the second motion sensor may be examples of accelerometer 1000 (e.g., MEMS voice accelerometer) or another inertial sensor (e.g., gyroscope).

Means for performing functionality at block 1920 may include a motion sensor system 1303 and/or other components of the apparatus as shown in FIG. 13.

At block 1930, the method 1900 may include obtaining a first optical signal reflected from the blood vessel measured by an optical sensor. In some embodiments, the first acoustic sensor, the second acoustic sensor, the first motion sensor, the second motion sensor, and the optical sensor may be contactable with a skin of the user. However, in some embodiments, the first acoustic sensor, the second acoustic sensor, the first motion sensor, the second motion sensor, and the optical sensor may not be contactable with a skin of the user.

In some embodiments, the method 1900 may further include determining one or more physiological characteristics of the blood vessel based at least on the first optical signal, the one or more physiological characteristics of the blood vessel comprising a diameter of the blood vessel, a distension of the blood vessel, volumetric blood flow, a pulse wave velocity of the blood vessel, motion of the blood vessel or tissue, or a combination thereof.

Means for performing functionality at block 1930 may include an optical sensor system 1301 and/or other components of the apparatus as shown in FIG. 13.

At block 1940, the method 1900 may include determining the physiological parameter of the user based on the first acoustic signal, the second acoustic signal, the first distance, the first motion signal, the second motion signal, the second distance, and the first optical signal.

In some embodiments, the method 1900 may further include obtaining a second optical signal reflected from the blood vessel measured by a second optical sensor, the optical sensor and the second optical sensor disposed at a third distance from each other. In some implementations, the method 1900 may further include determining a first characteristic of the blood vessel based on the first distance and a first temporal difference between a measurement time of the first acoustic signal and a measurement time of the second acoustic signal, the first characteristic comprising a pulse wave velocity determined based on the first temporal difference and the first distance; determining a second characteristic of the blood vessel based on the second distance and a second temporal difference between a measurement time of the first motion signal and a measurement time of the second motion signal, the second characteristic comprising a pulse wave velocity determined based on the second temporal difference and the second distance; and determining a third characteristic of the blood vessel based on the third distance and a third temporal difference between the a measurement time of the first optical signal and a measurement time of the second optical signal, the third characteristic comprising a pulse wave velocity determined based on the third temporal difference and the third distance. In some implementations, the method 1900 may further include determining the physiological parameter of the user based at least on a combination of one or more physiological characteristics of the blood vessel, the first characteristic of the blood vessel, and the second characteristic of the blood vessel. In some implementations, the physiological parameter of the user may include a blood pressure of the user.

In some embodiments, the method 1900 may further include providing, to a trained machine learning model, the reflected first optical signal, the first and second acoustic signals, the first and second motion signals, or a combination thereof; and obtaining, from the trained machine learning model, a prediction the physiological parameter of the user.

In some embodiments, the method 1900 may further include waking up the first motion sensor, the second motion sensor, or the optical sensor from a low-power state responsive to the first acoustic sensor or the second acoustic sensor detecting an acoustic signal having a signal quality above a threshold.

Means for performing functionality at block 1940 may include a control system 1306 and/or other components of the apparatus as shown in FIG. 13.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

Implementation examples are described in the following numbered clauses:

Clause 1: A multi-sensor user device comprising: a first acoustic sensor and a second acoustic sensor disposed at a first distance from each other, the first and second acoustic sensors each configured to obtain respective first and second acoustic signals associated with a blood vessel of a user, the first distance corresponding to a first characteristic of the blood vessel; a first motion sensor and a second motion sensor disposed at a second distance from each other, the first and second motion sensors each configured to obtain respective first and second motion signals associated with the blood vessel, the second distance corresponding to a second characteristic of the blood vessel; a photoacoustic sensor configured to obtain a photoacoustic signal generated from light incident on the blood vessel, the photoacoustic signal corresponding to one or more physiological characteristics of the blood vessel, wherein a combination of the one or more physiological characteristics of the blood vessel, the first characteristic of the blood vessel, and the second characteristic of the blood vessel correlate to a physiological parameter of the user; and a wearable structure securable to the user and comprising the first acoustic sensor, the second acoustic sensor, the first motion sensor, the second motion sensor, and the photoacoustic sensor.

Clause 2: The user device of clause 1, wherein the first acoustic sensor, the second acoustic sensor, the first motion sensor, the second motion sensor, and the photoacoustic sensor are contactable with a skin of the user.

Clause 3: The user device of any one of clauses 1-2 wherein the first distance corresponds to a first temporal difference between the respective first and second acoustic signals, the first characteristic comprising a pulse wave velocity determined based on the first temporal difference and the first distance; and the second distance corresponds to a second temporal difference between the respective first and second motion signals, the second characteristic comprising a pulse wave velocity determined based on the second temporal difference and the second distance.

Clause 4: The user device of any one of clauses 1-3 wherein the first and second acoustic sensors each comprise a microphone, and the first and second motion sensors each comprise an accelerometer.

Clause 5: The user device of any one of clauses 1-4 wherein the first motion sensor, the second motion sensor, or a combination thereof comprises a voice accelerometer configured to obtain motion signals.

Clause 6: The user device of any one of clauses 1-5 wherein the one or more physiological characteristics of the blood vessel comprise a diameter of the blood vessel, a distension of the blood vessel, volumetric blood flow, or a combination thereof.

Clause 7: The user device of any one of clauses 1-6 further comprising a control system, wherein the control system is configured to determine the physiological parameter of the user based at least on the combination of the one or more physiological characteristics of the blood vessel, the first characteristic of the blood vessel, and the second characteristic of the blood vessel.

Clause 8: The user device of any one of clauses 1-7 wherein the physiological parameter of the user comprises a blood pressure of the user.

Clause 9: The user device of any one of clauses 1-8 further comprising a control system, wherein the control system is configured to use a trained machine learning model configured to: receive the photoacoustic signal, the first and second acoustic signals, the first and second motion signals, or a combination thereof; and output a prediction the physiological parameter of the user.

Clause 10: The user device of any one of clauses 1-9 further comprising a control system, wherein the control system is configured to wake up the first motion sensor, the second motion sensor, or the photoacoustic sensor from a low-power state responsive to the first acoustic sensor or the second acoustic sensor detecting an acoustic signal having a signal quality above a threshold.

Clause 11: The user device of any one of clauses 1-10 further comprising a data interface configured to communicate with a control system, the control system configured to determine the physiological parameter of the user based on the combination of the one or more physiological characteristics of the blood vessel, the first characteristic of the blood vessel, and the second characteristic of the blood vessel.

Clause 12: A method of determining a physiological parameter of a user, the method comprising: obtaining a first acoustic signal associated with a blood vessel of the user measured by a first acoustic sensor of a wearable device, and a second acoustic signal associated with the blood vessel measured by a second acoustic sensor of the wearable device which is disposed at a first distance from the first acoustic sensor; obtaining a first motion signal associated with the blood vessel measured by a first motion sensor of the wearable device, and a second motion signal associated with the blood vessel measured by a second motion sensor of the wearable device which is disposed at a second distance from the first motion sensor; obtaining a photoacoustic signal generated from light incident on the blood vessel measured by a photoacoustic sensor; and determining the physiological parameter of the user based on the first acoustic signal, the second acoustic signal, the first distance, the first motion signal, the second motion signal, the second distance, and the photoacoustic signal.

Clause 13: The method of clause 12, wherein the first acoustic sensor, the second acoustic sensor, the first motion sensor, the second motion sensor, and the photoacoustic sensor are contactable with a skin of the user.

Clause 14: The method of any one of clauses 12-13 further comprising determining a physiological characteristic of the blood vessel based on: the first distance and a first temporal difference, the first temporal difference comprising a difference in a measurement time of the first acoustic signal and a measurement time of the second acoustic signal; the second distance and a second temporal difference, the second temporal difference comprising a difference in measurement time of the first motion signal and a measurement time of the second motion signal; or a combination thereof.

Clause 15: The method of any one of clauses 12-14 wherein the physiological characteristic of the blood vessel comprises a pulse wave velocity of the blood vessel.

Clause 16: The method of any one of clauses 12-15 wherein the determining of the physiological parameter of the user comprises determining a blood pressure of the user; and the determining of the blood pressure of the user is based on the pulse wave velocity of the blood vessel.

Clause 17: The method of any one of clauses 12-16 wherein the first and second acoustic sensors each comprise a microphone, and the first and second motion sensors each comprise an accelerometer.

Clause 18: The method of any one of clauses 12-17 further comprising determining one or more physiological characteristics of the blood vessel based on the photoacoustic signal; wherein the one or more physiological characteristics comprise a diameter of the blood vessel, a distension of the blood vessel, volumetric blood flow, or a combination thereof.

Clause 19: The method of any one of clauses 12-18 further comprising using a trained machine learning model configured to: receive the first acoustic signal, the second acoustic signal, the first motion signal, the second motion signal, the photoacoustic signal, or a combination thereof; and output a prediction the physiological parameter of the user.

Clause 20: The method of any one of clauses 12-19 further comprising waking up the first motion sensor, the second motion sensor, or the photoacoustic sensor from a low-power state responsive to the first acoustic sensor or the second acoustic sensor detecting an acoustic signal having a signal quality above a threshold.

Clause 21: An apparatus comprising: first acoustic sensing means and second acoustic sensing means disposed at a first distance from each other, the first and second acoustic sensing means each being for obtaining respective first and second acoustic signals associated with a blood vessel of a user, the first distance corresponding to a first characteristic of the blood vessel; first motion sensing means and second motion sensing means disposed at a second distance from each other, the first and second motion sensing means each being for obtaining respective first and second motion signals associated with the blood vessel, the second distance corresponding to a second characteristic of the blood vessel; photoacoustic sensing means for obtaining a photoacoustic signal generated from light incident on the blood vessel, the photoacoustic signal corresponding to one or more physiological characteristics of the blood vessel, wherein a combination of the one or more physiological characteristics of the blood vessel, the first characteristic of the blood vessel, and the second characteristic of the blood vessel correlate to a physiological parameter of the user; and wearable means for securing the apparatus to the user and comprising the first acoustic sensing means, the second acoustic sensing means, the first motion sensing means, the second motion sensing means, and the photoacoustic sensing means.

Clause 22: The apparatus of clause 21, wherein the first acoustic sensing means, the second acoustic sensing means, the first motion sensing means, the second motion sensing means, and the photoacoustic sensing means are contactable with a skin of the user.

Clause 23: The apparatus of any one of clauses 21-22 wherein the first distance corresponds to a first temporal difference between the respective first and second acoustic signals, the first characteristic comprising a pulse wave velocity determined based on the first temporal difference and the first distance; and the second distance corresponds to a second temporal difference between the respective first and second motion signals, the second characteristic comprising a pulse wave velocity determined based on the second temporal difference and the second distance.

Clause 24: The apparatus of any one of clauses 21-23 wherein the one or more physiological characteristics of the blood vessel comprise a diameter of the blood vessel, a distension of the blood vessel, volumetric blood flow, or a combination thereof.

Clause 25: The apparatus of any one of clauses 21-24 further comprising means for determining the physiological parameter of the user based at least on the combination of the one or more physiological characteristics of the blood vessel, the first characteristic, and the second characteristic.

Clause 26: The apparatus of any one of clauses 21-25 wherein the physiological parameter of the user comprises a blood pressure of the user.

Clause 27: A non-transitory computer-readable apparatus comprising a storage medium, the storage medium comprising a plurality of instructions configured to, when executed by one or more processors, cause an apparatus to: obtain a first acoustic signal associated with a blood vessel of a user measured by a first acoustic sensor of a wearable device, and a second acoustic signal associated with the blood vessel measured by a second acoustic sensor of the wearable device which is disposed at a first distance from the first acoustic sensor; obtain a first motion signal associated with the blood vessel measured by a first motion sensor of the wearable device, and a second motion signal associated with the blood vessel measured by a second motion sensor of the wearable device which is disposed at a second distance from the first motion sensor; obtain a photoacoustic signal generated from light incident on the blood vessel measured by a photoacoustic sensor; and determine a physiological parameter of the user based on the first acoustic signal, the second acoustic signal, the first distance, the first motion signal, the second motion signal, the second distance, and the photoacoustic signal.

Clause 28: The non-transitory computer-readable apparatus of clause 27, wherein the plurality of instructions are further configured to, when executed by the one or more processors, cause the apparatus to determine a physiological characteristic of the blood vessel based on: the first distance and a first temporal difference, the first temporal difference comprising a difference in a measurement time of the first acoustic signal and a measurement time of the second acoustic signal; the second distance and a second temporal difference, the second temporal difference comprising a difference in measurement time of the first motion signal and a measurement time of the second motion signal; or a combination thereof.

Clause 29: The non-transitory computer-readable apparatus of any one of clauses 27-28 wherein the determination of the physiological parameter of the user comprises determination of a blood pressure of the user based on the physiological characteristic of the blood vessel.

Clause 30: The non-transitory computer-readable apparatus of any one of clauses 27-29 wherein the plurality of instructions are further configured to, when executed by the one or more processors, cause the apparatus to determine one or more physiological characteristics of the blood vessel based on the photoacoustic signal; wherein the one or more physiological characteristics comprise a diameter of the blood vessel, a distension of the blood vessel, volumetric blood flow, or a combination thereof.

What is claimed:

1. A multi-sensor user device comprising:
a first acoustic sensor and a second acoustic sensor disposed at a first distance from each other, the first and second acoustic sensors each configured to obtain respective first and second acoustic signals associated with a blood vessel of a user, the first distance corresponding to a first characteristic of the blood vessel;
a first motion sensor and a second motion sensor disposed at a second distance from each other, the first and second motion sensors each configured to obtain respective first and second motion signals associated with the blood vessel, the second distance corresponding to a second characteristic of the blood vessel;
a photoacoustic sensor configured to obtain a photoacoustic signal generated from light incident on the blood vessel, the photoacoustic signal corresponding to one or more physiological characteristics of the blood vessel, wherein a combination of the one or more physiological characteristics of the blood vessel, the first characteristic of the blood vessel, and the second characteristic of the blood vessel correlate to a physiological parameter of the user; and
a wearable structure securable to the user and comprising the first acoustic sensor, the second acoustic sensor, the first motion sensor, the second motion sensor, and the photoacoustic sensor.

2. The user device of claim 1, wherein the first acoustic sensor, the second acoustic sensor, the first motion sensor, the second motion sensor, and the photoacoustic sensor are contactable with a skin of the user.

3. The user device of claim 1, wherein:
the first distance corresponds to a first temporal difference between the respective first and second acoustic signals, the first characteristic comprising a pulse wave velocity determined based on the first temporal difference and the first distance; and
the second distance corresponds to a second temporal difference between the respective first and second motion signals, the second characteristic comprising a pulse wave velocity determined based on the second temporal difference and the second distance.

4. The user device of claim 1, wherein the first and second acoustic sensors each comprise a microphone, and the first and second motion sensors each comprise an accelerometer.

5. The user device of claim 4, wherein the first motion sensor, the second motion sensor, or a combination thereof comprises a voice accelerometer configured to obtain motion signals.

6. The user device of claim 1, wherein the one or more physiological characteristics of the blood vessel comprise a diameter of the blood vessel, a distension of the blood vessel, volumetric blood flow, or a combination thereof.

7. The user device of claim 1, further comprising a control system, wherein the control system is configured to determine the physiological parameter of the user based at least on the combination of the one or more physiological characteristics of the blood vessel, the first characteristic of the blood vessel, and the second characteristic of the blood vessel.

8. The user device of claim 7, wherein the physiological parameter of the user comprises a blood pressure of the user.

9. The user device of claim 1, further comprising a control system, wherein the control system is configured to use a trained artificial intelligence model configured to:
receive the photoacoustic signal, the first and second acoustic signals, the first and second motion signals, or a combination thereof; and
output a prediction the physiological parameter of the user.

10. The user device of claim 1, further comprising a control system, wherein the control system is configured to wake up the first motion sensor, the second motion sensor, or the photoacoustic sensor from a low-power state responsive to the first acoustic sensor or the second acoustic sensor detecting an acoustic signal having a signal quality above a threshold.

11. The user device of claim 1, further comprising a data interface configured to communicate with a control system, the control system configured to determine the physiological parameter of the user based on the combination of the one or more physiological characteristics of the blood vessel, the first characteristic of the blood vessel, and the second characteristic of the blood vessel.

12. A method of determining a physiological parameter of a user, the method comprising:
obtaining a first acoustic signal associated with a blood vessel of the user measured by a first acoustic sensor of a wearable device, and a second acoustic signal associated with the blood vessel measured by a second acoustic sensor of the wearable device which is disposed at a first distance from the first acoustic sensor;
obtaining a first motion signal associated with the blood vessel measured by a first motion sensor of the wearable device, and a second motion signal associated with the blood vessel measured by a second motion sensor of the wearable device which is disposed at a second distance from the first motion sensor;
obtaining a photoacoustic signal generated from light incident on the blood vessel measured by a photoacoustic sensor; and
determining the physiological parameter of the user based on the first acoustic signal, the second acoustic signal, the first distance, the first motion signal, the second motion signal, the second distance, and the photoacoustic signal.

13. The method of claim 12, wherein the first acoustic sensor, the second acoustic sensor, the first motion sensor, the second motion sensor, and the photoacoustic sensor are contactable with a skin of the user.

14. The method of claim 12, further comprising determining a physiological characteristic of the blood vessel based on:
the first distance and a first temporal difference, the first temporal difference comprising a difference in a measurement time of the first acoustic signal and a measurement time of the second acoustic signal;
the second distance and a second temporal difference, the second temporal difference comprising a difference in measurement time of the first motion signal and a measurement time of the second motion signal; or
a combination thereof.

15. The method of claim 14, wherein the physiological characteristic of the blood vessel comprises a pulse wave velocity of the blood vessel.

16. The method of claim 15, wherein:
the determining of the physiological parameter of the user comprises determining a blood pressure of the user; and
the determining of the blood pressure of the user is based on the pulse wave velocity of the blood vessel.

17. The method of claim 12, wherein the first and second acoustic sensors each comprise a microphone, and the first and second motion sensors each comprise an accelerometer.

18. The method of claim 12, further comprising determining one or more physiological characteristics of the blood vessel based on the photoacoustic signal;
wherein the one or more physiological characteristics comprise a diameter of the blood vessel, a distension of the blood vessel, volumetric blood flow, or a combination thereof.

19. The method of claim 12, further comprising using a trained artificial intelligence model configured to:
receive the first acoustic signal, the second acoustic signal, the first motion signal, the second motion signal, the photoacoustic signal, or a combination thereof; and
output a prediction the physiological parameter of the user.

20. The method of claim 12, further comprising waking up the first motion sensor, the second motion sensor, or the photoacoustic sensor from a low-power state responsive to the first acoustic sensor or the second acoustic sensor detecting an acoustic signal having a signal quality above a threshold.

21. An apparatus comprising:
first acoustic sensing means and second acoustic sensing means disposed at a first distance from each other, the first and second acoustic sensing means each being for obtaining respective first and second acoustic signals associated with a blood vessel of a user, the first distance corresponding to a first characteristic of the blood vessel;
first motion sensing means and second motion sensing means disposed at a second distance from each other, the first and second motion sensing means each being for obtaining respective first and second motion signals associated with the blood vessel, the second distance corresponding to a second characteristic of the blood vessel;
photoacoustic sensing means for obtaining a photoacoustic signal generated from light incident on the blood vessel, the photoacoustic signal corresponding to one or more physiological characteristics of the blood vessel, wherein a combination of the one or more physiological characteristics of the blood vessel, the first characteristic of the blood vessel, and the second characteristic of the blood vessel correlate to a physiological parameter of the user; and
wearable means for securing the apparatus to the user and comprising the first acoustic sensing means, the second acoustic sensing means, the first motion sensing means, the second motion sensing means, and the photoacoustic sensing means.

22. The apparatus of claim 21, wherein the first acoustic sensing means, the second acoustic sensing means, the first motion sensing means, the second motion sensing means, and the photoacoustic sensing means are contactable with a skin of the user.

23. The apparatus of claim 21, wherein:
the first distance corresponds to a first temporal difference between the respective first and second acoustic signals, the first characteristic comprising a pulse wave velocity determined based on the first temporal difference and the first distance; and
the second distance corresponds to a second temporal difference between the respective first and second motion signals, the second characteristic comprising a pulse wave velocity determined based on the second temporal difference and the second distance.

24. The apparatus of claim 21, wherein the one or more physiological characteristics of the blood vessel comprise a diameter of the blood vessel, a distension of the blood vessel, volumetric blood flow, or a combination thereof.

25. The apparatus of claim 21, further comprising means for determining the physiological parameter of the user based at least on the combination of the one or more physiological characteristics of the blood vessel, the first characteristic, and the second characteristic.

26. The apparatus of claim 25, wherein the physiological parameter of the user comprises a blood pressure of the user.

27. A non-transitory computer-readable apparatus comprising a storage medium, the storage medium comprising a plurality of instructions configured to, when executed by one or more processors, cause an apparatus to:
obtain a first acoustic signal associated with a blood vessel of a user measured by a first acoustic sensor of a wearable device, and a second acoustic signal associated with the blood vessel measured by a second acoustic sensor of the wearable device which is disposed at a first distance from the first acoustic sensor;
obtain a first motion signal associated with the blood vessel measured by a first motion sensor of the wearable device, and a second motion signal associated with the blood vessel measured by a second motion sensor of the wearable device which is disposed at a second distance from the first motion sensor;
obtain a photoacoustic signal generated from light incident on the blood vessel measured by a photoacoustic sensor; and
determine a physiological parameter of the user based on the first acoustic signal, the second acoustic signal, the first distance, the first motion signal, the second motion signal, the second distance, and the photoacoustic signal.

28. The non-transitory computer-readable apparatus of claim 27, wherein the plurality of instructions are further configured to, when executed by the one or more processors, cause the apparatus to determine a physiological characteristic of the blood vessel based on:
the first distance and a first temporal difference, the first temporal difference comprising a difference in a measurement time of the first acoustic signal and a measurement time of the second acoustic signal;
the second distance and a second temporal difference, the second temporal difference comprising a difference in measurement time of the first motion signal and a measurement time of the second motion signal; or
a combination thereof.

29. The non-transitory computer-readable apparatus of claim 28, wherein the determination of the physiological parameter of the user comprises determination of a blood pressure of the user based on the physiological characteristic of the blood vessel.

30. The non-transitory computer-readable apparatus of claim 27, wherein the plurality of instructions are further configured to, when executed by the one or more processors, cause the apparatus to determine one or more physiological characteristics of the blood vessel based on the photoacoustic signal;
wherein the one or more physiological characteristics comprise a diameter of the blood vessel, a distension of the blood vessel, volumetric blood flow, or a combination thereof.
